(19) United States
(12) Patent Application Publication
(10) Pub. No.: US 2023/0210881 A1
(43) Pub. Date: Jul. 6, 2023

(54) NICOTINAMIDE MONONUCLEOTIDE DERIVATIVES FOR THE TREATMENT OF BACTERIAL INFECTIONS

(71) Applicant: NUVAMID SA, Epalinges (CH)

(72) Inventors: Guillaume BERMOND, Epalinges (CH); Laurent GARÇON, Epalinges (CH)

(73) Assignee: NUVAMID SA, Epalinges (CH)

(21) Appl. No.: 17/914,576

(22) PCT Filed: Mar. 26, 2021
(86) PCT No.: PCT/EP2021/057938

§ 371 Date: Sep. 26, 2022

(30) Foreign Application Priority Data
Mar. 27, 2020 (FR) FR2003085

(51) Int. Cl. A61K 31/7084; A61K 31/706; A61P 31/04

(52) U.S. Cl. CPC A61K 31/7084; A61K 31/706; A61P 31/04

(57) ABSTRACT

A compound of formula (I) or a pharmaceutically acceptable salt and/or solvate thereof;

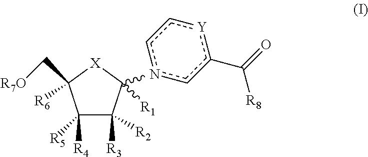

in which X, R₁, R₂, R₃, R₄, R₅, R₆, R₇, R₈, Y, [symbol] and [symbol] are as described in the claims, for the use thereof in the treatment of bacterial infections including those caused by caused by at least one bacterium of the genus selected from aerobic Gram-positive bacteria; Gram-negative enterobacteria; Gram-negative bacilli; Gram-negative anaerobic bacteria; Gram-positive anaerobic bacteria; mycobacteria and pathogens involved in sexually transmitted infections.

### FIELD OF THE INVENTION

The present invention relates to a compound of formula (I) or a pharmaceutically acceptable salt and/or solvate thereof for use in the treatment of bacterial infections.

### PRIOR ART

Bacterial infections are responsible for diseases or syndromes such as urinary tract infections, skin and soft tissue infections, sexually transmitted infections, tetanus, typhoid, tuberculosis, cholera, syphilis, salmonella, pneumonia, or sepsis (septicaemia). Despite the large number and diversity of antibacterial agents, bacterial infections are one of the leading causes of death in the world, especially in developing countries.

In the event of a bacterial infection, patients will usually receive antibiotics, but if appropriate treatment is delayed or if the bacteria are multiplied or the patients are infected with an antibiotic-resistant strain, the antibiotics will not be effective. In addition, the continued emergence of drug-resistant bacteria is a concern in both developed and developing countries.

Over-prescription of antibiotics appears to be one of the main reasons for the emergence of resistance. However, other factors such as the use of antibiotics in animal husbandry and the increasing number of antibacterial agents in cleaning products are also responsible for the development of resistance. In addition, even without exposure to antibiotics, DNA mutations and the acquisition of additional chromosomal DNA occur naturally in bacteria, which can lead to resistance.

There is therefore a need to develop new compounds active against wild-type bacteria but also against drug-resistant bacteria. These compounds should be able to overcome the resistance mechanisms developed by bacteria against currently used antibiotics and should have a maximum capacity of eradication of bacteria while presenting a low toxicity.

The NAD+/NADH redox couple is involved in hundreds of redox reactions in the cell, notably in mitochondria and the Kreps cycle. In addition, NAD+ is an essential cosubstrate for a number of non-redox enzymes (e.g., sirtuins (SIRTs), poly-ADP-rybosyl polymerases (PARPs), ADPR cyclases (ADP rybosyl cyclases such as CD38 and CD73), and mono-ADP-rybosyl transferases (MARTs) in mammals and DNA ligases and CobB/Sir2 family of deacetylase proteins in bacteria). It is thus necessary to maintain NAD+ homeostasis through active resynthesis and recycling of these degradation products.

For example, the sirtuin SIRT2 modulates many cellular processes including carcinogenesis, cell cycle, DNA damage and cellular response to infection. Upon infection with Listeria monocytogenes, SIRT2 is relocated from the cytoplasm to the nucleus where post-translational modifications on the enzyme (dephosphorylation) induce its association with chromatin and promote infection progression (2018, Cell Reports, 23, 1124-1137).

Furthermore, it was shown that increased intracellular NAD+ levels in endothelial cells infected with group A streptococci (GAS) resulted in the inhibition of intracellular GAS growth. This phenomenon was explained by the enhancement of autophagic clearance mechanisms (2020, Frontiers in Microbiology, 11, 117).

Increased oxidative stress caused by Helicobacter pylori infection alters intracellular calcium homeostasis in macrophages via “transient receptor potential melastatin-2” (TRPM2) (2017, Mucosal immunology, 10(2), 493-507). The more pronounced the increase in intracellular calcium concentration, such as in the absence of TRPM2, the more it results in an M1-like polarization of macrophages. This polarization leads to an increase in the inflammatory profile of the infection and a decrease in bacterial colonization. However, the mobilization of intracellular calcium reserves can also be done through the intermediary of cyclic ADP-ribose, the production of which is catalyzed by the enzyme ADP-ribose cyclase (CD38), of which NAD+ is a co-factor.

Alternatively, some bacteria, such as Mycobacterium tuberculosis, secrete necrotic exotoxins into the cytosol of infected macrophages. These exotoxins may have ADP-rybosyltransferase or β-NAD+ glycohydrolase activities. The latter deprive host cells, in the absence of other targets, of their NAD+ resources causing the activation of necroptosis mechanisms (2019, Journal of Biological Chemistry, 294(9), 3024-3036).

Nicotinamide mononucleotide (NMN) is a nucleotide that is best known for its role as an intermediate in nicotinamide adenine dinucleotide (NAD+) biosynthesis. Mainly known for its involvement in NAD+ biosynthesis, this particular molecule has been shown to be pharmacologically effective in several preclinical studies.

The aim of this invention is to propose an alternative to current treatments by providing nicotinamide mononucleotide derivatives for the treatment of bacterial infections.

The Applicant has observed that the derivatives of nicotinamide mononucleotide according to the invention are well-tolerated and can reduce bacterial propagation.

### SUMMARY

The present invention relates to a compound of formula (I)

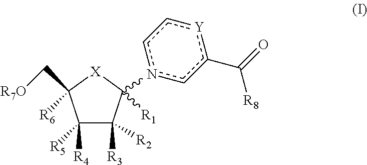

or a pharmaceutically acceptable salt and/or solvate thereof, wherein:
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl. C(O)O(C₁-C₁₂)-alkyl, C(O)-aryl, C(O)(C₁-C₁₂)-alkyl-(C₅-C₁₂)-aryl,
C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from P(O)R₉R₁₀, P(S)R₉R₁₀ and

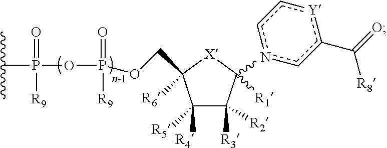

wherein
R₉ and R₁₀ are selected, independently of one another, from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, (C₅-C₁₂)-aryl-(C₁-C₈)-alkyl, (C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}C(O)R₁₂; wherein:
   R₁₁ is selected from C₁-C₁₀ alkyl, C₃-C₁₀ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁-C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; P(O)(OH)OP(O)(OH)₂; and an internal or external counter-ion;
   R₁₂ is selected from C₁-C₁₀alkyl, hydroxy, C₁-C₁₀alkoxy, C₂-C₈ alkenyloxy, C₂-C₈ alkynyloxy, halo(C₂-C₁₀)-alkoxy, C₃-C₁₀ cycloalkoxy, C₃-C₁₀ heterocycloalkyloxy, C₅-C₁₂ aryloxy, (C₁-C₄)-alkyl-(C₅-C₁₂)-aryloxy, (C₅-C₁₂)-aryl-(C₁-C₄)-alkyloxy and C₅-C₁₂ heteroaryloxy; wherein said aryl or heteroaryl groups are optionally substituted by one or two groups selected from halogen, trifluoromethyl, C₁-C₆ alkyl, C₁-C₆ alkoxy and cyano;
   R₁₃ and R₁₄ are selected independently from H, C₁-C₈ alkyl and (C₁-C₈)-alkyl-(C₅-C₁₂)-aryl;
   R_{α} and R_{α′} are selected independently from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen, nitro and cyano;
or R₉ and R₁₀ with the phosphorus atoms to which they are bonded, form a 6-member-ring, wherein —R₉-R₁₀— represents —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆ alkoxy and cyano;
X′ is selected from O, CH₂, S, Se, CHF, CF₂ and C═CH₂;
R_{1′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{8′} is selected from H, OR, NHR_{15′}, NR_{15′}R_{16′}, NH—NHR_{15′}, SH, CN, N₃ and halogen; wherein R_{15′} and R_{16′} are selected, independently of one another, from H, C₁-C₈ alkyl and C₁-C₈ alkyl-aryl;
Y′ selected from CH, CH₂, C(CH₃)₂ and CCH₃;
n is an integer selected from 1 to 3;
[symbol] represents a single or a double bond according to Y′; and
[symbol] represents the alpha or beta anomer according to the position of R_{1′};
R₈ is selected from H, OR, NHR₁₅, NR₁₅R₁₆, NH—NHR₁₅, SH, CN, N₃ and halogen; wherein R is selected from H and C₁-C₈ alkyl, and R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
Y is selected from CH, CH₂, C(CH₃)₂ and CCH₃;
[symbol] represents a single or a double bond according to Y; and
[symbol] represents the alpha or beta anomer according to the position of R₁,
   for use thereof in the treatment of bacterial infections.

According to an embodiment, in the formula (I):
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl aryl, C(O)NH(C₁-C₁₂)-alkyl aryl, C(O)O(C₁-C₁₂)-alkyl aryl and C(O)CHR_{AA}NH₂; wherein R_{AA} is a side-chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from H, P(O)R₉R₁₀ and P(S)R₉R₁₀; wherein R₉ and R₁₀ are selected, independently of one another, from OH, OR₁₁, C₁-C₈ alkyl, C₅-C₁₂ aryl and NHCHR_{AA}C(O)R₁₂; wherein:
   R₁₁ is selected from C₁-C₈ alkyl, C₅-C₁₂ aryl and P(O)(OH)OP(O)(OH)₂;
   R₁₂ is a C₁-C₈ alkyl; and
   R_{AA} is a side chain selected from a proteinogenic amino acid;
R₈ is selected from H, OR, NHR₁₃, NR₁₃R₁₄, NH—NHR₁₃, SH, CN, N₃ and halogen; wherein R₁₃ and R₁₄ are selected, independently of one another, from H, C₁-C₈ alkyl and C₁-C₈ alkyl-aryl;
Y is selected from CH, CH₂, C(CH₃)₂ and CCH₃;
[symbol] represents a single or a double bond according to Y; and
[symbol] represents the alpha or beta anomer according to the position of R₁.

In an embodiment, X represents oxygen.

In an embodiment, R₁ and R₆ each represent, independently of one another, hydrogen.

In an embodiment, R₂, R₃, R₄ and R₅ each represent, independently of one another, hydrogen or an OH.

In an embodiment, Y represents CH or CH₂.

In an embodiment, R₇ represents P(O)(OH)₂.

In an embodiment, R₇ represents

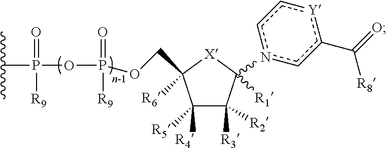

wherein;
R₉ is OH or OR₁₁, wherein R₁₁ is as defined in formula (I) and
X′ is oxygen;
R_{1′} and R_{6′} each represent hydrogen;
R_{2′}, R_{3′}, R_{4′} and R_{5′} are independently selected from hydrogen and OH;
R_{8′} is NH₂;
Y′ is selected from CH and CH₂;
n is equal to 2;
[symbol] represents a single or a double bond according to Y′; and
[symbol] represents the alpha or beta anomer according to the position de R_{1′}.

In an embodiment, R₇ represents P(O)(OH)₂.

In an embodiment, the compound of the invention is selected from the compounds of Table 1:

TABLE 1

| | |
|---|---|
| 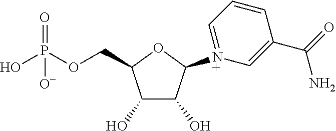 [SMILES: NC(=O)c1ccc[n+]([C@@H]2O[C@H](COP(=O)([O-])O)[C@@H](O)[C@H]2O)c1] | I-A |
| 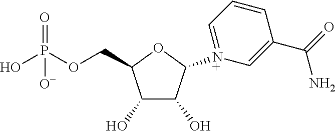 [SMILES: NC(=O)c1ccc[n+]([C@H]2O[C@H](COP(=O)([O-])O)[C@@H](O)[C@H]2O)c1] | I-B |
| 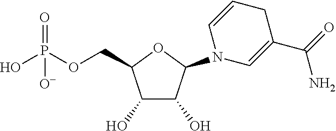 [SMILES: NC(=O)C1=CN([C@@H]2O[C@H](COP(=O)([O-])O)[C@@H](O)[C@H]2O)C=CC1] | I-C |
| 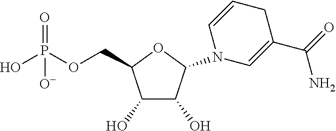 [SMILES: NC(=O)C1=CN([C@H]2O[C@H](COP(=O)([O-])O)[C@@H](O)[C@H]2O)C=CC1] | I-D |
| 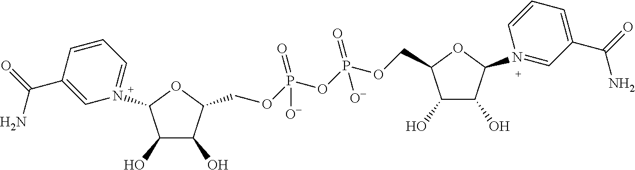 | I-G |
| 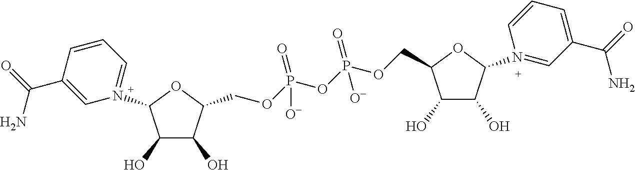 | I-H |
| 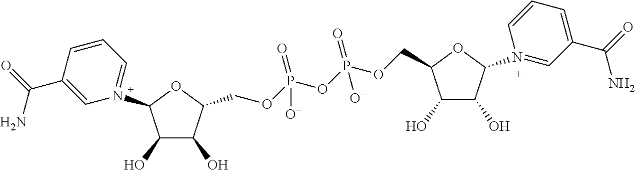 | I-I |
| 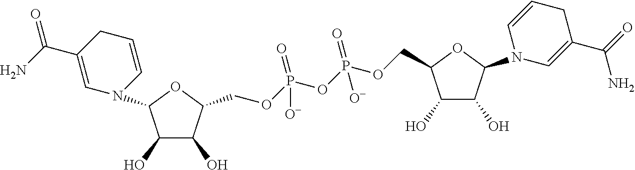 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] | I-J |
| 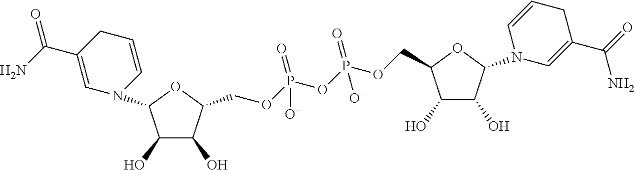 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] | I-K |
| 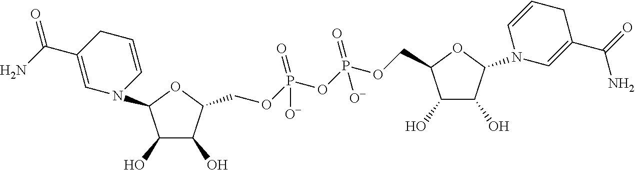 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] | I-L |

or a pharmaceutically acceptable salt and/or solvate thereof.

In one embodiment, the bacterial infection is caused by at least one bacterium of the genus selected from aerobic Gram-positive bacteria such as Streptococcus, Staphylococcus, Enterococcus or Bacillus; Gram-negative enterobacteria such as Escherichia coli, Klebsiella pneumonia, Enterobacter aerogenes, Enterobacter cloacae, Proteus vulgaris, Shigella flexneri, Serratia marcescens, Citrobacter freundii, Yersinia enterocolitica or Salmonella enteritidis; Gram-negative bacilli such as Pseudomonas aeruginosa, Acinetobacter baumannii, Burkholderia cepacia or Stenotrophomonas maltophilia; Gram-negative anaerobic bacteria such as Bacteroides, Fusobacterium or Eubacterium; Gram-positive anaerobic bacteria such as Propionibacterium, Peptococcus, Clostridium, Peptostreptococcus or Veillonella; mycobacteria such as Mycobacterium leprae or Mycobacterium tuberculosis; Helicobacter pylori and pathogens involved in sexually transmitted infections such as Neisseria, Haemophilus, Chlamydia or Mycoplasma.

In an embodiment, the bacterial infection is selected from bacterial skin and soft tissue infections, sexually transmitted bacterial infections, tetanus, typhoid, tuberculosis, cholera, diphtheria, syphilis, salmonella, pulmonary bacterial infections or sepsis.

In one embodiment, the bacterial infection is sepsis.

### Definitions

In the present invention the following terms have the following meaning.

Unless otherwise indicated, the nomenclature of the substituents which are not explicitly defined in the present invention is obtained by naming the terminal part of the functionality followed by the adjacent functionality towards the point of attachment.
“Alkyl” by itself or as part of another substituent, means a hydrocarbyl radical of formula CₙH₂ₙ₊₁ in which n is a number greater than or equal to 1. In general, the alkyl groups of this invention comprise 1 to 12 carbon atoms, preferably 1 to 10 carbon atoms, preferably 1 to 8 carbon atoms, more preferably 1 to 6 carbon atoms, even more preferably 1 to 2 carbon atoms. The alkyl groups can be linear or branched and can be substituted as indicated in the present invention.

Suitable alkyl groups include methyl, ethyl, n-propyl, i-propyl, n-butyl, i-butyl, s-butyl and t-butyl, pentyl and its isomers (for example, n-pentyl, iso-pentyl), hexyl and its isomers (for example, n-hexyl, iso-hexyl), heptyl and its isomers (for example, n-heptyl, iso-heptyl), octyl and its isomers (for example n-octyl, iso-octyl), nonyl and its isomers (for example, n-nonyl, iso-nonyl), decyl and its isomers (for example n-decyl, iso-decyl), undecyl and its isomers, dodecyl and its isomers. The preferred alkyl groups are the following: methyl, ethyl, n-propyl, i-propyl, n-butyl, i-butyl, s-butyl and t-butyl. The Cₓ-C_{y}-alkyls mean the alkyl groups which comprise from x to y carbon atoms.
“Alkenyl” by itself or as part of another substituent, means an unsaturated hydrocarbyl group, which may be linear or branched, comprising one or more carbon-carbon double bonds. Suitable alkenyl groups comprise between 2 and 12 carbon atoms, preferably between 2 and 8 carbon atoms, even more preferably between 2 and 6 carbon atoms. Non-limiting examples of alkenyl groups include ethenyl, 2-propenyl, 2-butenyl, 3-butenyl, 2-pentenyl and its isomers, 2-hexenyl and its isomers, 2,4-pentadienyl.
“Alkynyl” by itself or as part of another substituent, means a class of unsaturated monovalent hydrocarbyl groups, in which the unsaturation results from the presence of one or more carbon-carbon triple bonds. The alkynyl groups generally, and preferably, have the same number of carbon atoms as described above for the alkenyl groups. Non-limiting examples of alkynyl groups include ethynyl, 2-propynyl, 2-butynyl, 3-butynyl, 2-pentynyl and its isomers, 2-hexynyl and its isomers.
“Alkoxy” means an alkyl group as defined above, which is attached to another part by an oxygen atom. Examples of alkoxy groups include methoxy, isopropoxy, ethoxy, tert-butoxy and other groups. The alkoxy groups may optionally be substituted by one or more substituents. The alkoxy groups included in the compounds of this invention can optionally be substituted by a solubilising group.
“Aryl”, as it is used here, means an aromatic, polyunsaturated hydrocarbyl group having a single ring (for example phenyl) or a plurality of aromatic rings that are fused together (for example naphtyl) or covalently bonded, generally containing 5 to 12 atoms, preferably 6 to 10, at least one ring of which is aromatic. The aromatic ring can optionally comprise one or two additional rings (cycloalkyl, heterocyclyl or heteroaryl) which are fused thereto. The aryl is also intended to include the partially hydrogenated derivatives of the carbocyclic systems listed here. The aryl examples comprise phenyl, biphenyl, biphenylenyl, the 5- or 6-tetralinyl, naphtalene-1- or -2-yl, 4-, 5-, 6 or 7-indenyl, 1-2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphtyl, 1,2,3,4-tetrahydronaphtyl, 1,4-dihydronaphtyl, 1-, 2-, 3-, 4- or 5-pyrenyl.
“Alkylaryl” means an aryl group substituted by an alkyl group.
“Arylalkyl” means an alkyl group substituted by an aryl group.
“Aryloxy” means an O-aryl group, wherein the aryl is defined according to the present invention.
“Amino add” means an alpha-aminated carboxylic acid, in other words a molecule comprising a functional carboxylic acid group and a functional amine group in the alpha position of the carboxylic acid group, for example a proteinogenic amino acid or a non-proteinogenic amino acid such as 2-aminoisobutyric acid.
“Proteinogenic amino acid” means an amino acid which is incorporated in the proteins during the translation of the messenger RNA by the ribosomes in living organisms, in other words alanine (ALA), arginine (ARG), asparagine (ASN), aspartate (ASP), cysteine (CYS), glutamate (glutamic acid) (GLU), glutamine (GLN), glycine (GLY), histidine (HIS), isoleucine (ILE), leucine (LEU), lysine (LYS), methionine (MET), phenylalanine (PHE), proline (PRO), pyrrolysine (PYL), selenocysteine (SEL), serine (SER), threonine (THR), tryptophan (TRP), tyrosine (TYR) or valine (VAL).
“Non-proteinogenic amino acid” means an amino acid which is not naturally encoded or is not found in the genetic code of a living organism. Non-limiting examples of non-proteinogenic amino acids include ornithine, citrulline, argininosuccinate, homoserine, homocysteine, cysteine-sulfinic acid, 2-aminomuconic acid, S-aminolevulinic acid, β-alanine, cystathionine, γ-aminobutyrate, DOPA, δ-hydroxytryptophan, D-serine, ibotenic acid, α-aminobutyrate, 2-aminoisobutyrate, D-leucine, D-valine, D-alanine or D-glutamate.
“cycloalkyl” by itself or as part of another substituent means a cyclical alkyl, alkenyl or alkynyl group, in other words a saturated or unsaturated, monovalent hydrocarbyl group, having 1 or 2 cyclic structures. Cycloalkyl includes monocyclic or bicyclic hydrocarbyl groups. The cycloalkyl groups may comprise 3 carbon atoms or more in the ring and generally, according to this invention, comprise 3 to 10, preferably 3 to 8, yet more preferably 3 to 6 carbon atoms. Non-limiting examples of cycloalkyl groups include cyclopropyl, cyclobutyl, cyclopentyl and cyclohexyl; cyclopropyl being particularly preferred.
“Cycloalkyloxy” means an O-cycloalkyl group, wherein the cycloalkyl is defined according to the present invention.
“Halogen” or “halo” means fluoro, chloro, bromo or iodo. The preferred halo groups are fluoro and chloro.
“Haloalkyl” alone or in combination, means an alkyl radical having the meaning as defined above, wherein one or more hydrogen atoms are replaced by a halogen as defined above. Examples of such halogenoalkyl radicals include chloromethyl, 1-bromoethyl, fluoromethyl, difluoromethyl, trifluoromethyl, the 1,1,1-trifluoroethyl and similar radicals. Cₓ-C_{y}-haloalkyl and halo-(Cₓ-C_{y})-alkyl mean haloalkyl groups which comprise from x to y carbon atoms. The preferred halogenoalkyl groups are difluoromethyl and trifluoromethyl.
“Heteroalkyl” means an alkyl group as defined above, in which one or more carbon atoms are replaced by a heteroatom selected from the atoms of oxygen, nitrogen and sulfur. In the heteroalkyl groups, the heteroatoms are only bonded to carbon atoms along the alkyl chain, in other words each heteroatom is separated from any other heteroatom by at least one carbon atom. However, the heteroatoms of nitrogen and sulfur can optionally be oxidised and the heteroatoms of nitrogen can optionally be quaternised. A heteroalkyl is bonded to another group or to another molecule only via a carbon atom, in other words the bond atom is not selected from the heteroatoms included in the heteroalkyl group.
“Heteroaryl” by itself or as part of another substituent means aromatic rings having 5 to 12 carbon atoms or systems containing 1 to 2 rings that are fused or covalently bonded, typically containing 5 to 6 atoms; at least one of the rings being aromatic; wherein one or more carbon atoms in one or more rings is replaced by one or more oxygen, nitrogen and/or sulfur atoms; the heteroatoms of nitrogen and sulfur can optionally be oxidised and the heteroatoms of nitrogen can optionally be quaternised. Such rings can be fused with an aryl, cycloalkyl, heteroaryl or heterocyclyl ring. Non-limiting examples of heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyel, 2,1,3-benzothiadiazolyl, thiénopyridinyl, purinyl, imidazol[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is called “heterocycloalkyl” or “heterocycyl”.
“Heteroaryloxy” means an —O-heteroaryl group, wherein the heteroaryl is defined according to the present invention.
“Heterocycyl”, “heterocycloalkyl” or “heterocyclo” by itself or as part of another substituent means fully saturated or partially unsaturated, cyclic, non-aromatic groups, (for example, monocycle with 3 to 7 members, bicycle with 7 to 11 members, or comprising a total of 3 to 10 atoms in the ring) which have at least one heteroatom in at least one ring containing carbon atoms. Each ring of the heterocyclyl group comprising a heteroatom can have 1, 2, 3 or 4 heteroatoms selected from nitrogen, oxygen and/or sulfur atoms, nitrogen and sulfur heteroatoms can optionally be oxidised and the nitrogen heteroatoms can optionally be quaternised. Each carbon atom of the heterocycle can be substituted by oxo (for example piperidone, pyrrolidinone). The heterocyclic group can be attached to any carbon atom or heteroatom of the ring or of the cyclic system, when the valence allows it. The rings of the multi-cyclic heterocycles can be fused, bridged and/or joined by one or more spiro atoms. Non-limiting examples of heterocyclic groups include oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl,
tetrahydroisoquinolin-4-yl, thiomorpholin-4-yl, thiomorpholin-4-ylulfoxide, thiomorpholin-4-ylsulfone, 1,3-dioxolanyl, 1,4-oxathianyl, 1H-pyrrolizinyl, tetrahydro-1,1-dioxothiophenyl, N-formylpiperazinyl, and morpholin-4-yl.
“Heterocycloalkyloxy” means an —O-heterocyclyl group, wherein the heterocyclyl is defined according to the present invention.
The term “substituent” or “substituted” means that a hydrogen radical on a compound or a group is replaced by any desired group which is substantially stable under the reaction conditions in an unprotected form or when it is protected by a protective group. The examples of preferred substituents are those found in the compounds and embodiments presented here, as well as the halogeno, alkyl or aryl groups as defined above, hydroxyl, alkoxy groups as defined above, nitro, thiol, heterocycloalkyl, heteroaryl, cyano, cycloalkyl groups as defined above, as well as a solubilising group, —NRR′, —NR—CO—R′, —CONRR′, —SO₂NRR′, where R and R′ are each independently selected from hydrogen and the alkyl, cycloalkyl, aryl, heterocycloalkyl or heteroaryl groups as defined above.
The asymmetric carbon bonds can be represented here by using a solid triangle ( [symbol] ), a dotted triangle ( [symbol] ) or a zigzag line ( [symbol] ).
The term “pharmaceutically acceptable excipient” refers to an inert carrier or support used as a solvent or diluent, in which the pharmaceutically active agent is formulated and/or administered, and which does not produce an undesirable, allergic or other reaction when it is administered to an animal, preferably a human being. This includes all solvents, dispersion media, coatings, antibacterial and antifungal agents, isotonic agents, absorption retardants and other similar ingredients. For human administration, the preparations must meet the standards of sterility, general safety and purity, as required by the regulating offices, such as the FDA or EMA, for example. Within the meaning of the invention, “pharmaceutically acceptable excipients” includes all pharmaceutically acceptable excipients as well as all pharmaceutically acceptable supports, diluents, and/or additives.
The “pharmaceutically acceptable salts” comprise the acid and base addition salts of these salts. Suitable acid addition salts are formed from acids which form non-toxic salts. This includes, for example, acetate, adipate, aspartate, benzoate, besylate, bicarbonate/carbonate, bisulfate/sulfate, borate, camsylate, citrate, cyclamate, edisylate, esylate, formiate, fumarate, gluceptate, gluconate, glucuronate, hexafluorophosphate, hibenzate, chlorhydrate/chloride, bromhydrate/bromide, hydroiodide/iodide, isethionate, lactate, malate, maleate, malonate, mesylate, methylsulfate, naphtylate, 2-napsylate, nicotinate, nitrate, orotate, oxalate, palmitate, pamoate, phosphate/hydrogenophosphate/dihydrogenophosphate, pyroglutamate, saccharate, stearate, succinate, tannate, tartrate, tosylate, trifluoroacetate and xinofoate salts. Suitable basic salts are formed from bases which form non-toxic salts. These include, for example the salts of aluminium, arginine, benzathine, calcium, choline, diethylamine, diolamine, glycine, lysine, magnesium, meglumine, olamine, potassium, sodium, tromethamine, 2-(diethylamino)ethanol, ehanolamine, morpholine, 4-(2-hydroxyethyl)morpholine and zinc. Acid and base hemisalts can also be formed, for example, hemisulfates and chemical calcium salts. The preferred pharmaceutically acceptable salts are the chlorhydrate/chloride, bromide/hydrobromide, bisulfate/sulfate, nitrate, citrate and acetate.

The pharmaceutically acceptable salts can be prepared by one or more of these methods:
(i) by reacting the compound with the desired acid;
(ii) by reacting the compound with the desired base;
(iii) by removing a protective labile group in an acid or base medium of a suitable precursor of the compound or by opening the ring of a suitable cyclic precursor, for example a lactone or a lactam, using the desired acid; or
iv) by transforming one salt of the compound into another, by reaction with a suitable acid or by means of a suitable ion-exchange column.

All these reactions are generally carried out in solution. The salt can precipitate from the solution and be collected by filtration or can be recovered by evaporation of the solvent. The degree of ionisation of the salt can vary from completely ionised to almost unionised.
“Pharmaceutically acceptable” means approved or able to be approved by a regulating body or included in a known pharmacopoeia for use in animals, and more preferably in humans. It may be a substance that is not biologically or otherwise undesirable, in other words the substance can be administered to an individual without causing undesirable biological effects or deleterious interactions with one of the components of the composition in which it is contained.
“Solvate” is used here to describe a molecular complex comprising the compound of the invention and one or more molecules of pharmaceutically acceptable solvent, for example ethanol.
The term “administration”, or a variant of this term (for example, “to administer”), means delivering the active agent or active substance, alone or in a pharmaceutically acceptable composition, to the patient for whom the symptom or disease must be treated or prevented.
The term “bacterial Infection” refers to an infection caused by one or more types of bacteria.
The term “subject” refers to a mammal, preferably a human. According to the present invention, a subject is a mammal, preferably a human, suffering from a bacterial infection. According to an embodiment, the subject is a “patient”, i.e. a mammal, preferably a human, who is waiting to receive, or who is receiving medical care or who was/is/will be the subject of a medical procedure, or who is monitored for the development of a bacterial infection.
The term “human” refers to a subject of both sexes and at any stage of development (in other words newborn, infant, juvenile, adolescent, adult).
The term “therapeutically effective quantity” (or more simply an “effective quantity”) as used here refers to the quantity of active agent or active ingredient which is targeted, without causing significant negative or undesirable side effects for the subject needing treatment, prevention, reduction, relief or slowing (attenuation) of one or more symptoms or manifestations of a bacterial infection and/or of one or more complications associated with bacterial infections.
The terms “to treat” or “treatment”, as used here, mean a therapeutic treatment, a prophylactic (or preventive) treatment, or both a therapeutic treatment and a prophylactic (or preventive) treatment, wherein the aim is to prevent, reduce, relieve and/or slow (attenuate) one or more symptoms or manifestations of a bacterial infection and/or prevent, reduce, relieve and/or slow (attenuate) one or more complications associated with bacterial infections, in a subject having need thereof.

The symptoms and manifestations of bacterial infections comprise, but are not limited to, a more or less high fever, pain and other symptoms depending on the organ involved: for example, pharyngeal pain, cough, respiratory discomfort, abdominal pain, diarrhea or vomiting. According to one embodiment, a manifestation of a bacterial infection is the presence of bacteria in one or more biological fluids (such as urine, blood, sputum) and/or in one or more organs of the subject.

Complications associated with bacterial infections usually involve a worsening of the disease or the development of new signs, symptoms or pathological changes that may spread throughout the body and affect organs other than those initially affected and may lead to the development of new diseases resulting from an already existing disease. The complication may also occur as a result of various treatments. According to one embodiment, “complications associated with bacterial infections” refers to, but is not limited to, sepsis or bacteraemia associated with sepsis, local or generalized inflammation, septic shock, encephalitis, meningitis, meningoencephalitis, peritonitis, pericarditis, endocarditis, septic arthritis, rheumatic fever, urinary tract infection, pyelonephritis, disseminated intravascular coagulation (DIC), pancreatitis, splenomegaly and hepatitis.

In an embodiment, “to treat” or “treatment” refers to a therapeutic treatment. In another embodiment, “to treat” or “treatment” refers to a prophylactic or preventive treatment. In yet another embodiment, “to treat” or “treatment” means both a prophylactic (or preventive) treatment and a therapeutic treatment.

In an embodiment, the aim of the treatment according to the present invention is to cause at least one of the following elements:
(a) an improvement in the clinical condition of the patient, in particular a reduction or disappearance of fever and pain associated with the affected organs;
(b) a decrease in the bacterial load in the affected organ(s) and/or in the body fluids;
(c) containment of the bacterial infection to the affected organ in order to avoid bacterial proliferation leading to a generalized infection;
(d) prevention of one or more complications associated with bacterial infections.

### DETAILED DESCRIPTION

The present invention therefore relates to the use of nicotinamide mononucleotide derivatives for the treatment of bacterial infections.

Compounds for Treating or Prevent the Propagation of Bacterial Infections.

The present invention relates to a compound of formula (I)

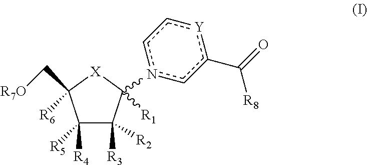

or a pharmaceutically acceptable salt and/or solvate thereof, wherein:
   X is selected from O, CH₂, S, Se, CHF, CF₂ and C═CH₂;
   R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O(C₁-C₁₂)-alkyl, C(O)NH(C₁-C₁₂)-alkyl. C(O)O(C₁-C₁₂)-alkyl. C(O)-aryl, C(O)(C₁-C₁₂)-alkyl-(C₅-C₁₂)-aryl,
   C(O)NH(C₁-C₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
   R₆ is selected from H, azido, cyano, C₁-C₈ alkyl, C₁-C₈ thio-alkyl, C₁-C₈ heteroalkyl and OR; wherein R is selected from H and C₁-C₈ alkyl;
   R₇ is selected from P(O)R₉R₁₀, P(S)R₉R₁₀ and

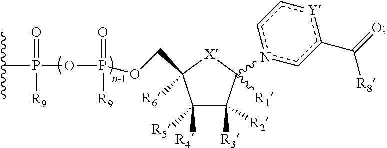

wherein
   R₉ and R₁₀ are selected, independently of one another, from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, (C₅-C₁₂)-aryl-(C₁-C₈)-alkyl, (C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}C(O)R₁₂; wherein:
      R₁₁ is selected from C₁-C₁₀ alkyl, C₃-C₁₀ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁-C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; P(O)(OH)OP(O)(OH)₂; and an internal or external counter-ion;
      R₁₂ is selected from C₁-C₁₀alkyl, hydroxy, C₁-C₁₀ alkoxy, C₂-C₈ alkenyloxy, C₂-C₈ alkynyloxy, halo(C₂-C₁₀)-alkoxy, C₃-C₁₀ cycloalkoxy, C₃-C₁₀ heterocycloalkyloxy, C₅-C₁₂ aryloxy, (C₁-C₄)-alkyl-(C₅-C₁₂)-aryloxy, (C₅-C₁₂)-aryl-(C₁-C₄)-alkyloxy and C₅-C₁₂ heteroaryloxy; wherein said aryl or heteroaryl groups are optionally substituted by one or two groups selected from halogen, trifluoromethyl, C₁-C₆ alkyl, C₁-C₆ alkoxy and cyano;
      R₁₃ and R₁₄ are selected independently from H, C₁-C₈ alkyl and (C₁-C₈)-alkyl-(C₅-C₁₂)-aryl;
      R_{α} and R_{α′} are selected independently from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen, nitro and cyano;
   or R₉ and R₁₀, with the phosphorus atoms to which they are bonded, form a 6-member-ring, wherein —R₉-R₁₀— represents —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆alkoxy and cyano;
   X′ is selected from O, CH₂, S, Se, CHF, CF₂ and C═CH₂;
   R_{1′} is selected from H, azido, cyano, C₁-C₈ alkyl, C₁-C₈ thio-alkyl, C₁-C₈ heteroalkyl and OR; wherein R is selected from H and C₁-C₈ alkyl;
   R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
   R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
   R_{8′} is selected from H, OR, NHR_{15′}, NR_{15′}R_{16′}, NH—NHR_{15′}, SH, CN, N₃ and halogen; wherein R_{15′} and R_{16′} are selected, independently of one another, from H, C₁-C₈ alkyl and C₁-C₈ alkyl-aryl;
   Y′ selected from CH, CH₂, C(CH₃)₂ and CCH₃;
   n is an integer selected from 1 to 3;
   [symbol] represents a single or a double bond according to Y′; and
   [symbol] represents the alpha or beta anomer according to the position of R_{1′};
   R₈ is selected from H, OR, NHR₁₅, NR₁₅R₁₆, NH—NHR₁₅, SH, CN, N₃ and halogen; wherein R is selected from H and C₁-C₈ alkyl, and R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
   Y is selected from CH, CH₂, C(CH₃)₂ and CCH₃;
   [symbol] represents a single or a double bond according to Y; and
   [symbol] represents the alpha or beta anomer according to the position of R₁,
for use thereof in the treatment of bacterial infections.
According to an embodiment, in the formula (I):
   X is selected from O, CH₂, S, Se, CHF, CF₂ and C═CH₂;
   R₁ is selected from H, azido, cyano, C₁-C₈ alkyl, C₁-C₈ thio-alkyl, C₁-C₈ heteroalkyl and OR; wherein R is selected from H and C₁-C₈ alkyl;
   R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl aryl, C(O)NH(C₁-C₁₂)-alkyl aryl, C(O)O(C₁-C₁₂)-alkyl aryl and C(O)CHR_{AA}NH₂; wherein R_{AA} is a side-chain selected from a proteinogenic amino acid;
   R₆ is selected from H, azido, cyano, C₁-C₈ alkyl, C₁-C₈ thio-alkyl, C₁-C₈ heteroalkyl and OR; wherein R is selected from H and C₁-C₈alkyl;
   R₇ is selected from H, P(O)R₉R₁₀ and P(S)R₉R₁₀; wherein R₉ and R₁₀ are selected, independently of one another, from OH, OR₁₁, C₁-C₈ alkyl, C₅-C₁₂ aryl and NHCHR_{AA}C(O)R₁₂; wherein:
      R₁₁ is selected from C₁-C₈ alkyl, C₅-C₁₂ aryl and P(O)(OH)OP(O)(OH)₂;
      R₁₂ is a C₁-C₈ alkyl; and
      R_{AA} is a side chain selected from a proteinogenic amino acid;
   R₈ is selected from H, OR, NHR₁₃, NR₁₃R₁₄, NH—NHR₁₃, SH, CN, N₃ and halogen; wherein R₁₃ and R₁₄ are selected, independently of one another, from H, C₁-C₈ alkyl and C₁-C₈ alkyl-aryl;
   Y is selected from CH, CH₂, C(CH₃)₂ and CCH₃;
   [symbol] represents a single or a double bond according to Y; and
   [symbol] represents the alpha or beta anomer according to the position of R₁.

According to an embodiment, the compound of formula (I) is not N-ribosylnicotinamide of formula:

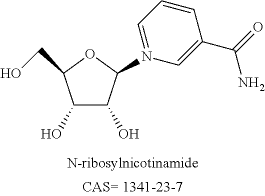

According to an embodiment, the compound of formula (I) is not a salt and/or solvate of N-ribosylnicotinamide.

According to an embodiment, X is selected from O, CH₂ and S. In a preferred embodiment, X is oxygen.

According to an embodiment, R₁ and R₆ each represent, independently of one another, hydrogen or OH. In an embodiment, R₁ and R₆ each represent hydrogen.

According to an embodiment, R₁ is selected from hydrogen or OH. In an embodiment, R₁ is OH. In an embodiment, R₁ is hydrogen.

According to an embodiment, R₂, R₃, R₄ and R₅ are selected, independently of one another, from H, halogen, hydroxyl, C₁-C₁₂ alkyl and OR; wherein R is as defined above. In a preferred embodiment, R₂, R₃, R₄ and R₅ are selected, independently of one another, from H, hydroxyl and OR; wherein R is as defined above. In a more preferred embodiment, R₂, R₃, R₄ and R₅ are selected, independently of one another, from hydrogen or OH.

According to an embodiment, R₂ and R₃ are identical. In an embodiment. R₂ and R₃ are identical and represent OH. In an embodiment, R₂ and R₃ are identical and represent hydrogen.

According to an embodiment, R₂ and R₃ are different. In a preferred embodiment, R₂ is hydrogen and R₃ is OH. In a more preferred embodiment, R₂ is OH and R₃ is hydrogen.

According to an embodiment, R₄ and R₅ are identical. In an embodiment, R₄ and R₅ are identical and represent OH. In an embodiment, R₄ and R₅ are identical and represent hydrogen.

According to an embodiment, R₄ and R₅ are different. In a preferred embodiment, R₄ is OH and R₅ is hydrogen. In a more preferred embodiment, R₄ is hydrogen and R₅ is OH.

According to an embodiment, R₃ and R₄ are different. In an embodiment, R₃ is OH and R₄ is hydrogen. In an embodiment, R₃ is hydrogen and R₄ is OH.

According to an embodiment, R₃ and R₄ are identical. In a preferred embodiment. R₃ and R₄ are identical and represent OH. In a more preferred embodiment, R₃ and R₄ are identical and represent hydrogen.

According to an embodiment, R₂ and R₅ are different. In an embodiment, R₂ is hydrogen and R₅ is OH. In an embodiment, R₂ is OH and R₅ is hydrogen.

According to an embodiment, R₂ and R₅ are identical. In a preferred embodiment, R₂ and R₅ are identical and represent hydrogen. In a more preferred embodiment, R₂ and R₅ are identical and represent OH.

According to an embodiment, R₆ is selected from hydrogen or OH. In an embodiment, R₆ is OH. In a preferred embodiment, R₆ is hydrogen.

According to an embodiment, R₇ is selected from hydrogen, P(O)R₉R₁₀ and

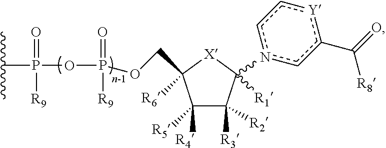

wherein R₉, R₁₀, R_{1′}-R_{6′}, R_{8′}, X′, Y′, n, [symbol] and [symbol] are as described above.

According to an embodiment, R₇ is selected from P(O)R₉R₁₀ or P(S)R₉R₁₀; wherein R₉ and R₁₀ are as defined above. In a preferred embodiment, R₇ is P(O)R₉R₁₀; wherein R₉ and R₁₀ are as defined above. In a more preferred embodiment, R₇ is P(O)(OH)₂.

In another embodiment, R₇ is

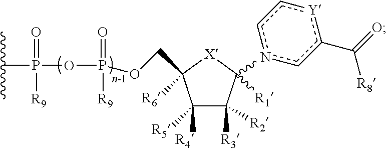

wherein R₉, R_{1′}, R_{2′}, R_{3′}, R_{4′}, R_{5′}, R_{6′}, R_{8′}, X′, Y′, n, [symbol] and [symbol] are as defined above.

In a particular embodiment, R₇ is

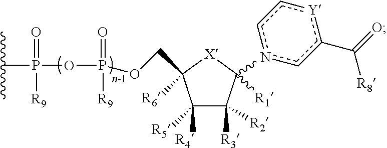

wherein:
R₉ is as defined above, preferably R₉ is OH or OR₁₁, wherein R₁₁ is as defined above, more preferably R₉ is OH
X′ is selected from O, CH₂ and S; preferably X′ is oxygen;
R_{1′} is selected from H and OH, preferably R_{1′} is H;
R_{2′}, R_{3′}, R_{4′} and R_{5′} are selected, independently of one another, from H, halogen, hydroxyl, C₁-C₁₂ alkyl and OR; wherein R is as defined above; preferably R_{2′}, R_{3′}, R_{4′} and R_{5′} are selected, independently of one another, from H and OH;
R_{6′} is selected from H or OH; preferably R_{6′} is H;
R_{8′} is selected from H, OR, NHR_{15′} or NR_{15′}R_{16′}, wherein R_{15′} and R_{16′} are as described above; preferably R_{8′} is NH₂;
Y′ is selected from CH or CH₂;
n is an integer selected from 1 to 3; preferably n is equal to 2;
[symbol] represents a single or a double bond according to Y′; and
[symbol] represents the alpha or beta anomer according to the position of R_{1′};

According to an embodiment, n is equal to 1. According to an embodiment, n is equal to 2. According to an embodiment, n is equal to 3.

In an embodiment, R₇ is not hydrogen.

In an embodiment, R₈ is selected from H, OR, NHR₁₅ and NR₁₅R₁₆; wherein R₁₅ and R₁₆ are as defined above. In a preferred embodiment, R₈ is NHR₁₅; wherein R₁₅ is as defined above. In a preferred embodiment, Re is NH₂.

In an embodiment, Y is CH. In an embodiment, Y is CH₂.

In a preferred embodiment, the compounds of formula (I) are compounds of formula (I-1):

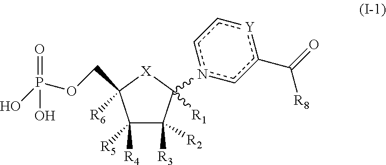

or a pharmaceutically acceptable salt and/or solvate thereof, wherein R₁, R₂, R₃, R₄, R₅, R₆, R₈, X, Y, [symbol] and [symbol] are as defined above for the compounds of formula (I).

In a preferred embodiment, the compounds of formula (I) are compounds of formula (I-2):

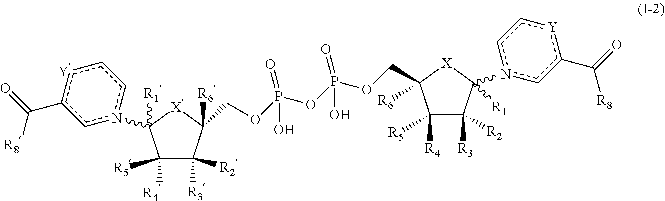

or a pharmaceutically acceptable salt and/or solvate thereof, wherein R₁, R₂, R₃, R₄, R₅, R₆, R₈, X, Y, R₁′, R₂′, R₃′, R₄′, R₅′, R₆′, R₈′, X′, Y′, [symbol] and [symbol] are as defined above for the compounds of formula (I).

In a preferred embodiment, the compounds of formula (I) are those in which X represents oxygen.

In a preferred embodiment, among the compounds of formula (I), the invention also relates to a compound of formula (II):

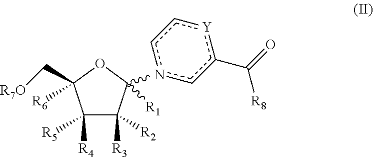

or a pharmaceutically acceptable salt and/or solvate thereof, wherein R₁, R₂, R₃, R₄, R₅, R₆, R₇, R₈, Y, [symbol] and [symbol] are as defined above for the compounds of formula (I).

In a preferred embodiment, the compounds of formula (II) are compounds of formula (II-1):

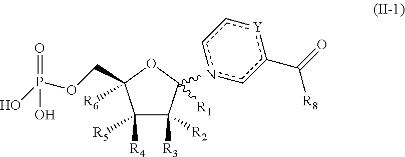

or a pharmaceutically acceptable salt and/or solvate thereof, wherein R₁, R₂, R₃, R₄, R₅, R₆, R₈, Y, [symbol] and [symbol] are as defined above for the compounds of formula (I).

In a preferred embodiment, the compounds of formula (II) are compounds of formula (II-2):

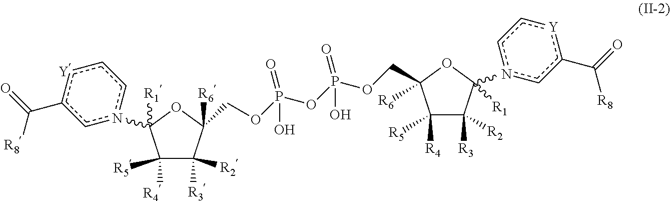

or a pharmaceutically acceptable salt and/or solvate thereof, wherein R₁, R₂, R₃, R₄, R₅, R₆, R₈, Y, R₁′, R₂′, R₃′, R₄′, R₅′, R₆′, R₈′, Y′, [symbol] and [symbol] are as defined above for the compounds of formula (I).

In a preferred embodiment, the compounds of formula I are those in which R₁ is hydrogen.

In a preferred embodiment, among the compounds of formula (I), the invention also relates to a compound of formula (III):

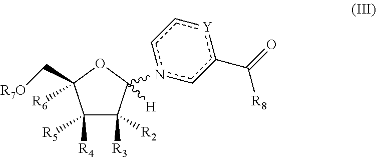

or a pharmaceutically acceptable salt and/or solvate thereof, wherein R₂, R₃, R₄, R₅, R₆, R₇, R₈, Y, [symbol] and [symbol] are as defined above for the compounds of formula (I).

In a preferred embodiment, the compounds of formula (III) are compounds of formula (III-1):

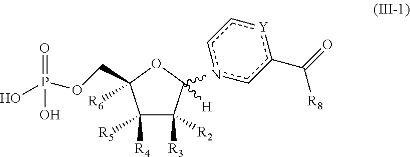

or a pharmaceutically acceptable salt and/or solvate thereof, wherein R₂, R₃, R₄, R₅, R₆, R₈, Y, [symbol] and [symbol] are as defined above for the compounds of formula (I).

In a preferred embodiment, the compounds of formula (III) are compounds of formula (III-2):

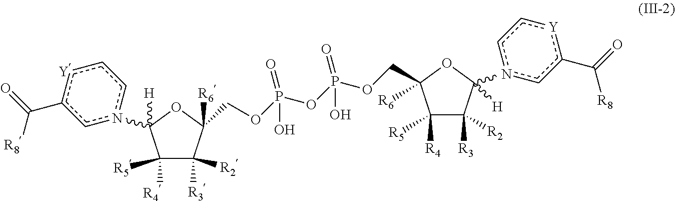

or a pharmaceutically acceptable salt and/or solvate thereof, wherein R₂, R₃, R₄, R₅, R₆, R₈, Y, R₂′, R₃′, R₄′, R₅′, R₆′, R₈′, Y′, [symbol] and [symbol] are as defined above for the compounds of formula (I).

In a preferred embodiment, the compounds of formula (I) are those in which R₂ is OH and R₃ is hydrogen.

In a preferred embodiment, the compounds of formula (I) are those in which R₄ is hydrogen and R₅ is OH.

In a preferred embodiment, the compounds of formula (I) are those in which R₃ and R₄ are identical and represent hydrogen.

In a preferred embodiment, among the compounds of formula (I), the invention also relates to a compound of formula (IV):

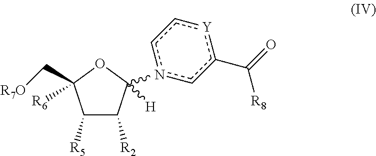

or a pharmaceutically acceptable salt and/or solvate thereof, wherein R₂, R₅, R₆, R₇, R₈, Y, [symbol] and [symbol] are as defined above for the compounds of formula (I).

In a preferred embodiment, the compounds of formula (IV) are compounds of formula (IV-1):

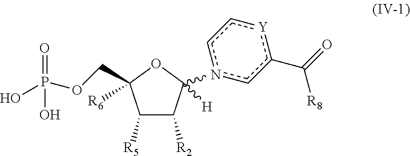

or a pharmaceutically acceptable salt and/or solvate thereof, wherein R₂, R₅, R₆, R₈, Y, [symbol] and [symbol] are as defined above for the compounds of formula (I).

In a preferred embodiment, the compounds of formula (IV) are compounds of formula (IV-2):

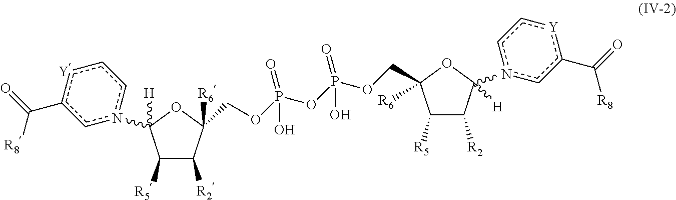

or a pharmaceutically acceptable salt and/or solvate thereof, wherein R₂, R₅, R₆, R₈, Y, R₂′, R₅′, R₆′, R₈′, Y′, [symbol] and [symbol] are as defined above for the compounds of formula (I).

In a preferred embodiment, the compounds of formula (I) are those in which R₂ and R₅ are identical and represent OH.

In a preferred embodiment, among the compounds of formula (I), the invention also relates to a compound of formula (V):

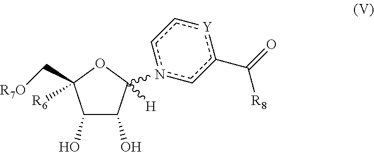

or a pharmaceutically acceptable salt and/or solvate thereof, wherein R₆, R₇, R₈, Y, [symbol] and [symbol] are as defined above for the compounds of formula (I).

In a preferred embodiment, the compounds of formula (V) are compounds of formula (V-1):

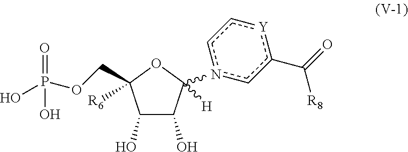

or a pharmaceutically acceptable salt and/or solvate thereof, wherein R₆, R₈, Y, [symbol] and [symbol] are as defined above for the compounds of formula (I).

In a preferred embodiment, the compounds of formula (V) are compounds of formula (V-2):

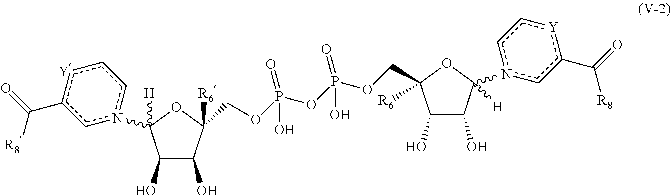

or a pharmaceutically acceptable salt and/or solvate thereof, wherein R₆, R₈, Y, R₆′, R₈′, Y′, [symbol] and [symbol] are as defined above for the compounds of formula (I).

In a preferred embodiment, the compounds of formula (I) are those in which R₆ is hydrogen.

In a preferred embodiment, among the compounds of formula (I), the invention also relates to a compound of formula (VI):

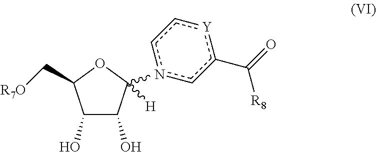

or a pharmaceutically acceptable salt and/or solvate thereof, wherein R₇, R₈, Y, [symbol] and [symbol] are as defined above for the compounds of formula (I). In a preferred embodiment, the compounds of formula (I) are those in which R₈ is NH₂.

In a preferred embodiment, the compounds of formula (VI) are compounds of formula (VI-1):

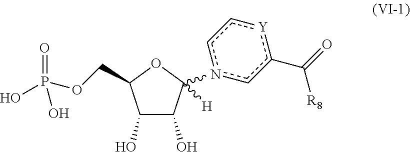

or a pharmaceutically acceptable salt and/or solvate thereof, wherein R₈, Y, [symbol] and [symbol] are as defined above for the compounds of formula (I).

In a preferred embodiment, the compounds of formula (VI) are compounds of formula (VI-2):

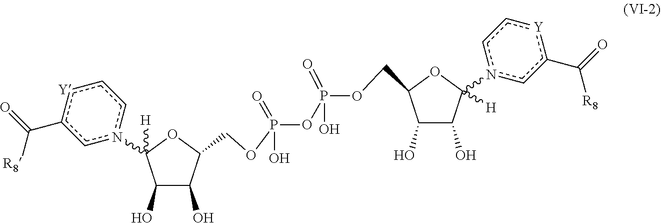

or a pharmaceutically acceptable salt and/or solvate thereof, wherein R₈, Y, R₈′, Y′, [symbol] and [symbol] are as defined above for the compounds of formula (I).

In a preferred embodiment, among the compounds of formula (I), the invention also relates to a compound of formula (VII):

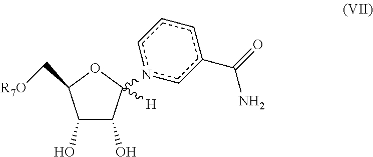

or a pharmaceutically acceptable salt and/or solvate thereof, wherein R₇, Y, [symbol] and [symbol] are as defined above for the compounds of formula (I). With the proviso that when R₇ is hydrogen, Y is CH₂.

In a preferred embodiment, the compounds of formula (VII) are compounds of formula (VII-1):

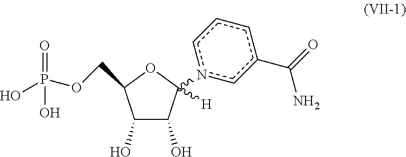

or a pharmaceutically acceptable salt and/or solvate thereof, wherein Y, [symbol] and [symbol] are as defined above for the compounds of formula (I).

In a preferred embodiment, the compounds of formula (VII) are compounds of formula (VII-2):

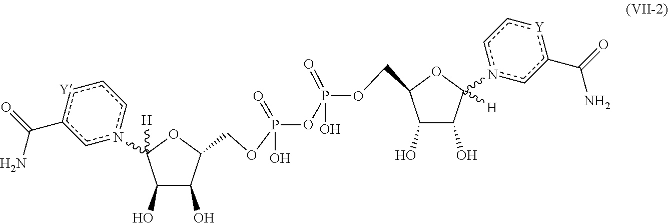

or a pharmaceutically acceptable salt and/or solvate thereof, wherein Y, Y′, [symbol] and [symbol] are as defined above for the compounds of formula (I).

In a preferred embodiment, the compounds of formula (I) are those in which Y is CH.

In a preferred embodiment, among the compounds of formula (I), the invention also relates to a compound of formula (VIII):

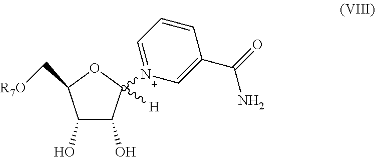

or a pharmaceutically acceptable salt and/or solvate thereof, wherein R₇ and [symbol] are as defined above for the compounds of formula (I). With the proviso that R₇ is not hydrogen.

In a preferred embodiment, the compounds of formula (VIII) are compounds of formula (VIII-1):

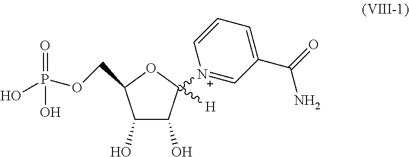

or a pharmaceutically acceptable salt and/or solvate thereof, wherein [symbol] is as defined above for the compounds of formula (I).

In a preferred embodiment, the compounds of formula (VIII) are compounds of formula (VIII-2):

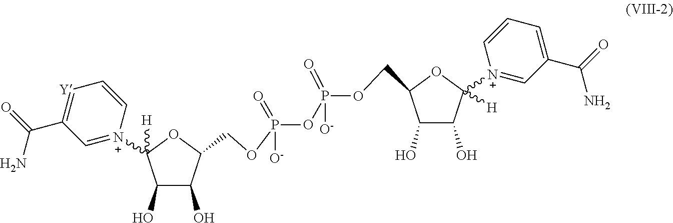

or a pharmaceutically acceptable salt and/or solvate thereof, wherein [symbol] is as defined above for the compounds of formula (I).

In a preferred embodiment, the compounds of formula (I) are those in which Y is CH₂.

In a preferred embodiment, among the compounds of formula (I), the invention also relates to a compound of formula (IX):

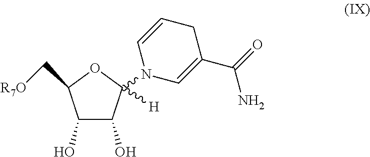

or a pharmaceutically acceptable salt and/or solvate thereof, wherein R₇ and [symbol] are as defined above for the compounds of formula (I).

In a preferred embodiment, the compounds of formula (IX) are compounds of formula (IX-1):

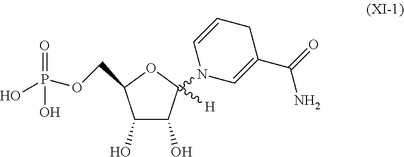

or a pharmaceutically acceptable salt and/or solvate thereof, wherein [symbol] is as defined above for the compounds of formula (I).

In a preferred embodiment, the compounds of formula (IX) are compounds of formula (IX-2):

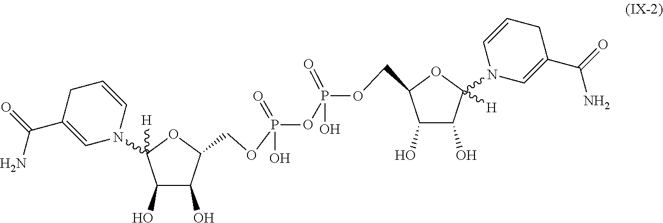

or a pharmaceutically acceptable salt and/or solvate thereof, wherein [symbol] is as defined above for the compounds of formula (I).

According to an embodiment, the compounds of the invention are selected from the compounds of Table 2 below or a pharmaceutically acceptable salt and/or solvate thereof:

TABLE 2

| Compounds | |
|---|---|
| (anomers) | Structure |
| I-A (beta) | 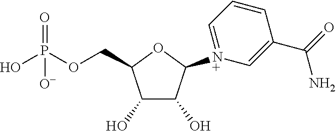 [SMILES: NC(=O)c1ccc[n+]([C@@H]2O[C@H](COP(=O)([O-])O)[C@@H](O)[C@H]2O)c1] |
| I-B (alpha) | 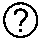 |
| I-C (beta) | 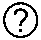 |
| I-D (alpha) | 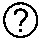 |
| I-E (beta) | 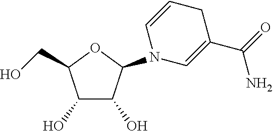 [SMILES: NC(=O)C1=CN([C@@H]2O[C@H](CO)[C@@H](O)[C@H]2O)C=CC1] |
| I-F (alpha) | 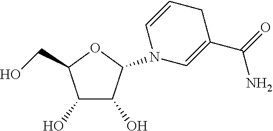 [SMILES: NC(=O)C1=CN([C@H]2O[C@H](CO)[C@@H](O)[C@H]2O)C=CC1] |
| I-G (beta, beta) | 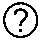 |
| I-H (beta, alpha) | 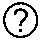 |
| I-I (alpha, alpha) | 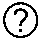 |
| I-J (beta, beta) | 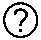 |
| I-K (beta, alpha) | 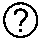 |
| I-L (alpha, alpha) | 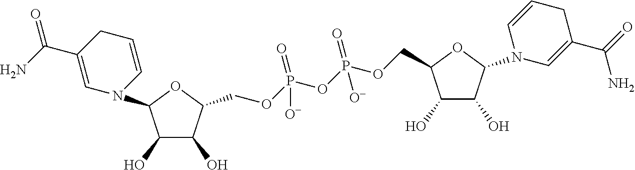 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

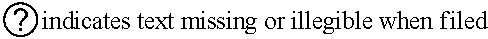

[symbol]

[symbol]

Depending on the conditions, especially the pH conditions, under which the compounds of the invention are found, some atoms may be in ionized form, especially some OH may be in O⁻ form, and vice versa.

In a preferred embodiment, the compounds of the invention are the compounds of formula I-A to I-D of Table 2 above or a pharmaceutically acceptable salt and/or solvate thereof.

In a preferred embodiment, the compound of the invention is the compound of formula I-A or I-B of Table 2 above or a pharmaceutically acceptable salt and/or solvate thereof.

In a preferred embodiment, the compound of the invention is the compound of formula I-A or I-C of Table 2 above or a pharmaceutically acceptable salt and/or solvate thereof.

In a more preferred embodiment, the compound of the invention is the compound of formula I-A or a pharmaceutically acceptable salt and/or solvate thereof.

In a preferred embodiment, the compounds of the invention are the compounds of formula I-G to I-L of Table 2 above or a pharmaceutically acceptable salt and/or solvate thereof.

In a preferred embodiment, the compounds of the invention are the compounds of formula I-G, I-H and I-I of Table 2 above or a pharmaceutically acceptable salt and/or solvate thereof.

In a preferred embodiment, the compounds of the invention are the compounds of formula I-A, I-B, I-G, I-H and I-I of Table 2 above or a pharmaceutically acceptable salt and/or solvate thereof.

Pharmaceutical Composition for Treating Bacterial Infections

According to another embodiment, the present invention relates to a pharmaceutical composition comprising at least one compound of the invention and at least one pharmaceutically acceptable excipient.

According to another embodiment, the present invention relates to a drug comprising at least one compound of the invention.

In an embodiment, the pharmaceutical composition of the invention or the drug of the invention comprises, in addition of at least one compound of the invention as active substances, additional therapeutic agents and/or active substances. Non-limiting examples of additional therapeutic agents and/or active substances comprise antibiotics, antibacterial agents, anti-inflammatory agents.

Method

According to another aspect, the invention relates to a method for preparing compounds of formula (I) as described above.

In particular, the compounds of formula (I) disclosed herein can be prepared as described below from substrates A-E. A person skilled in the art would understand that these reaction schemes are in no way limiting and variations can be made without departing from the spirit and scope of the present invention.

According to an embodiment, the invention relates to a method for preparing compounds of formula (I) as described above.

The method involves, in a first step, the mono-phosphorylation of a compound of formula (A), in the presence of phosphoryl chloride and trialkyl phosphate, to lead to the phosphorodichloridate of formula (B),

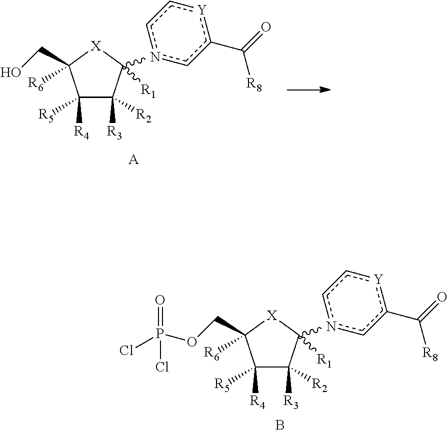

wherein X, R₁, R₂, R₃, R₄, R₅, R₆, R₈, Y, [symbol] and [symbol] are as defined above for the compounds of formula (I).

In a second step, the phosphorodichloridate of formula (B) is hydrolysed to lead to the phosphate of formula (C),

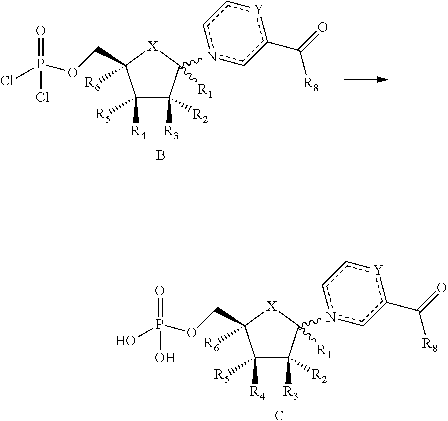

wherein X, R₁, R₂, R₃, R₄, R₅, R₆, R₈, Y, [symbol] and [symbol] are as defined above for the compounds of formula (I).

According to an embodiment, the dimeric compounds wherein R7 is

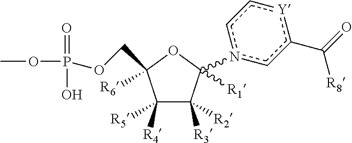

can be obtained by reacting an intermediate of formula (C) as described above with an intermediate phosphorodichloridate of formula (B′), obtained under the same conditions as the intermediates of formula (B):

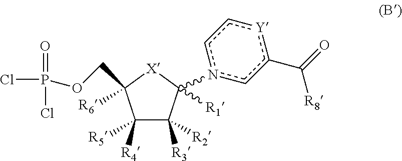

wherein R₁′, R₂′, R₃′, R₄′, R₅′, R₆′, R₈′, X′, Y′, [symbol] and [symbol] are as defined above.

According to an embodiment, the compound of formula (A) is synthesised using various methods known to a person skilled in the art.

According to an embodiment, the compound of formula (A) is synthesised by reacting the pentose of formula (D) with a nitrogenous derivative of formula (E), wherein R, R₂, R₃, R₄, R₅, R₆, R₇, Y are as described above for the compounds of formula I, leading to the compound of formula (A-1) which is then selectively deprotected in order to give the compound of formula (A),

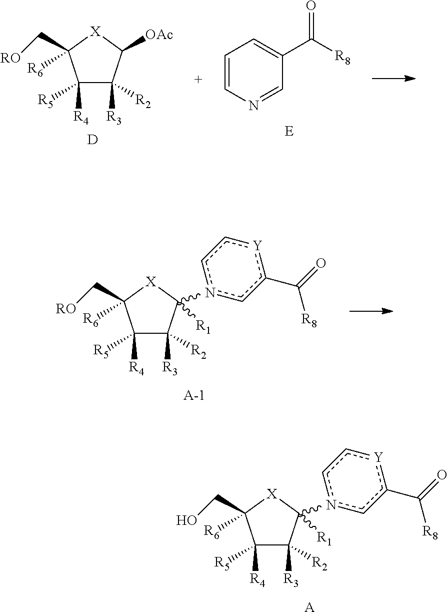

wherein X, R₁, R₂, R₃, R₄, R₅, R₆, R₈, Y, [symbol] and [symbol] are as defined above for the compounds of formula (I).

According to an embodiment, R is a suitable protective group known to a person skilled in the art. In an embodiment, the protective group is selected from triarylmethyls and/or silyls. Non-limiting examples of triarylmethyl include the trityl, monomethoxytrityl, 4,4′-dimethoxytrityl and 4,4′,4″-trimethoxytrityl groups. Non-limiting examples of silyl groups comprise the trimethylsilyl, tert-butyldimethylsilyl, triisopropylsilyl, tert-butyldiphenylsilyl, tri-iso-propylsilyloxymethyl and [2-(trimethylsilyl)ethoxy]methyl groups.

According to an embodiment, any hydroxyl group attached to the pentose is protected by a suitable protective group known to a person skilled in the art.

The choice and exchange of protective groups is within the skill of a person skilled in the art. The protective groups can also be removed by methods that are well-known to a person skilled in the art, for example with an acid (for example, a mineral or organic acid), a base or fluoride source.

In a preferred embodiment, the nitrogenous derivative of formula (E) is coupled to the pentose of formula (D) by a reaction in the presence of a Lewis acid leading to the compound of formula (A-1). Non-limiting examples of Lewis acids include TMSOTf, BF₃·OEt₂, TiCl₄ and FeCl₃.

In an embodiment, the method of the present invention further comprises a step of reducing the compound of formula (A) by various methods that are well known to a person skilled in the art, leading to the compound of formula (A′) wherein X is CH₂, and R₁, R₂, R₃, R₄, R₅, R₆, R₈, Y, [symbol] and [symbol] are as defined above for the compounds of formula (I).

In a particular embodiment, the present invention relates to a method for preparing compounds of formula I-A to I-D.

In a first step, the nicotinamide of formula E is coupled to the ribose tetraacetate of formula D by a coupling reaction in the presence of a Lewis acid, leading to the compound of formula A-1:

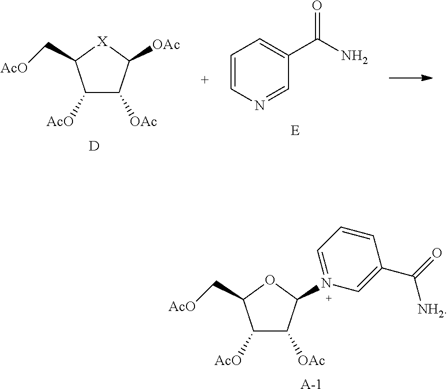

In a second step, an ammonia treatment of the compound of formula A-1 is carried out, leading to the compound of formula A-2:

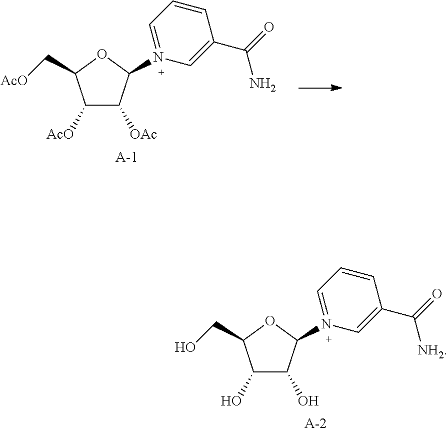

In a third step, the mono-phosphorylation of the compound of formula A-2, in the presence of phosphoryl chloride and a trialkyl phosphate, leads to the phosphorodichloridate of formula A-3:

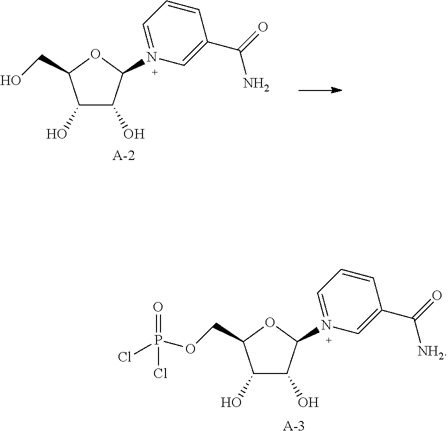

In a fourth step, the phosphorodichloridate of formula A-3 is hydrolysed in order to give the compound of formula I-A:

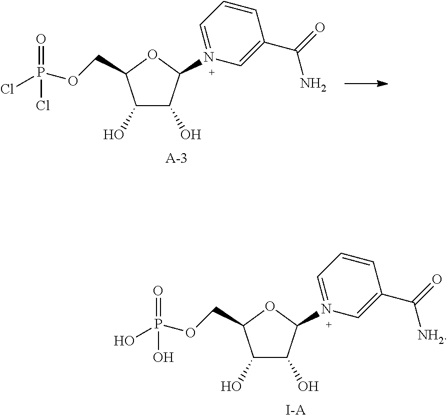

In an embodiment, a step of reducing the compound of formula A-2 is carried out, leading to the compound of formula I-E.

The compound of formula I-E is then monophosphorylated as described for the fourth step and hydrolysed in order to give the compound of formula I-C.

Use

The present invention thus relates to the compounds of the invention for use thereof in the treatment of bacterial infections.

Without wishing to be bound by any theory, the compounds of the invention allow the treatment of bacterial infections by a mechanism of polarization of the host macrophages resulting from the mobilization of intracellular calcium reserves via ADP-ribose cyclase. In another mechanism, the compounds of the invention allow the replenishment of internal NAD+ reserves following the action of exotoxins with β-NAD+ glycohydrolase activities.

According to an embodiment, the present invention relates to compounds of formula (I)-(IX) or a pharmaceutically acceptable salt and/or solvate thereof, as described above, for use thereof in the treatment of bacterial infections.

In an embodiment, the present invention relates to compounds of formula (I)-(IX) or a pharmaceutically acceptable salt and/or solvate thereof, as described above, for use thereof in the prophylactic treatment of bacterial infections.

In an embodiment, the present invention relates to compounds of formula (I)-(IX) or a pharmaceutically acceptable salt and/or solvate thereof, as described above, for use thereof in the treatment of an infection caused by at least one Gram-negative or Gram-positive bacterium.

In an embodiment, the present invention relates to compounds of formula (I)-(IX) or a pharmaceutically acceptable salt and/or solvate thereof, as described above, for use thereof in the prophylactic treatment of an infection caused by at least one bacterium of the genus selected from, but not limited to:
Gram-positive aerobic bacteria such as Staphylococcus aureus, Streptococcus pneumoniae, Enterococcus faecalis, Bacillus anthracis, Staphylococcus epidermidis, or Streptococcus pyogenes;
Gram-negative enterobacteria such as Escherichia coli, Klebsiella pneumonia, Enterobacter aerogenes, Enterobacter cloacae, Proteus vulgaris, Shigella flexneri, Serratia marcescens, Citrobacter freundii, Yersinia enterocolitica or Salmonella enteritidis;
Gram-negative bacilli such as Pseudomonas aeruginosa, Acinetobacter baumannii, Burkholderia cepacia or Stenotrophomonas maltophilia;
Gram-negative anaerobic bacteria such as Bacteroides fragilis, Bacteroides distasonis, Bacteroides thetaiotaomicron, Bacteroide vulgatus, Fusobacterium mortiferum, Fusobacterium necrophorum. Fusobacterium varium, Eubacterium lentum;
Gram-positive anaerobic bacteria such as Propionibacterium acens, Clostridium difficile, Clostridium perfringens, Clostridium ramosum, Peptostreptococcus anaerobius, Peptostreptococcus micros, or Veillonella parvula;
Mycobacteria such as Mycobacterium leprae, Mycobacterium tuberculosis complex such as Mycobacterium tuberculosis and non-tuberculous mycobacteria such as Mycobacterium chelonae, Mycobacterium avium, Mycobacterium abscessus, Mycobacterium fortuitum, Mycobacterium malmoense, Mycobacterium gordonae, Mycobacterium terrae, Mycobacterium nonchromogenicium, Mycobacterium simiae, Mycobacterium scrofulaceum, Mycobacterium phlei, Mycobacterium xenopi, Mycobacterium marinum, or Mycobacterium ulcerans;
Helicobacter pylori and pathogens involved in sexually transmitted infections such as Neisseria gonorrhoeae, Haemophilus ducreyi, Chlamydia trachomatis or Mycoplasma genitallium.

Some bacteria such as Salmonella, Legionella and Mycobacterium, in particular Mycobacteria tuberculosis, possess the ability to remain alive in macrophages.

In one embodiment, the compounds of the invention are capable of entering macrophages and having bactericidal activity therein.

In an embodiment, the present invention relates to compounds of formula (I)-(IX) or a pharmaceutically acceptable salt and/or solvate thereof, as described above, for use thereof in the treatment of infections with, but not limited to, Escherichia coli, pseudomonas, Haemophilus influenzae, Campylobacter, Enterococcus, Pneumococcus or Streptococcus.

In an embodiment, the present invention relates to compounds of formula (I)-(IX) or a pharmaceutically acceptable salt and/or solvate thereof, as described above, for use thereof in the treatment of a bacterial infection, selected from bacterial urinary tract infections, bacterial skin and soft tissue infections, sexually transmitted bacterial infections, tetanus, typhoid, tuberculosis, cholera, diphtheria, syphilis, salmonella, meningitis, sore throat, sinusitis, bronchitis, bacterial lung infections or sepsis.

In an embodiment, the present invention relates to compounds of formula (I)-(IX) or a pharmaceutically acceptable salt and/or solvate thereof, as described above, for use thereof in the treatment of sepsis.

In an embodiment, the present invention relates to compounds of formula (I)-(IX) or a pharmaceutically acceptable salt and/or solvate thereof, as described above, for use thereof in the treatment of pulmonary bacterial infections such as pneumonia, e.g. pneumonia caused by Escherichia coli, Haemophilus influenzae, Staphylococcus aureus, Streptococcus pneumoniae (causing pneumococcal pneumonia), Mycoplasma pneumoniae (causing mycoplasma pneumonia), Chlamydia pneumoniae, and/or Legionella pneumophila (responsible for Legionnaires' disease)

According to another embodiment, the present invention relates to a pharmaceutical composition comprising at least one compound of the invention, and at least one pharmaceutically acceptable excipient for use thereof in the treatment of bacterial infections.

According to another embodiment, the present invention relates to a drug comprising at least one compound of the invention for use thereof in the treatment of bacterial infections.

In an embodiment, the pharmaceutical composition of the invention or the drug of the invention comprises, in addition of at least one compound of the invention as active substances, additional therapeutic agents and/or active substances. Non-limiting examples of additional therapeutic agents and/or active substances comprise antibiotics, antibacterial agents, anti-inflammatory agents.

According to an embodiment, the present invention relates to the use of the compounds of the invention as described above for the treatment of bacterial infections. In an embodiment, the present invention relates to the use of the compounds of the invention as described above for the prophylactic treatment of bacterial infections.

According to another embodiment, the present invention relates to the use of a pharmaceutical composition comprising at least one compound of the invention and at least one pharmaceutically acceptable excipient for the treatment of bacterial infections.

According to another embodiment, the present invention relates to the use of a drug comprising at least one compound of the invention for the treatment of bacterial infections.

In an embodiment, the present invention relates to the use of the compounds of the invention as described above for the manufacture of a drug for the treatment of bacterial infections.

The present invention also relates to a method for treating bacterial infections in a subject in need thereof, said method comprising administering to said subject a therapeutically effective quantity of at least one compound or a composition of the invention as described above.

In an embodiment, the subject who is in need of a therapeutic or preventive treatment is diagnosed by a health professional. In practice, bacterial infections are diagnosed by any examination routinely performed in the medical environment, notably by a direct diagnosis, i.e. isolation of the bacteria in culture media, or an indirect diagnosis, by the detection of antibodies specific to the infection.

Preferably, the subject is a warm-blooded animal, more preferably a human.

According to an embodiment, the compounds of the invention can be administered within the framework of a combined therapy in which one or more compounds of the invention or a composition or a drug which contains a compound of the present invention, as active substances, are co-administered in combination with additional therapeutic agents and/or active substances. Non-limiting examples of additional therapeutic agents and/or active substances comprise antibiotics, antibacterial agents, anti-inflammatory agents.

In the above-described embodiments, the compound of the invention and other therapeutic active agents can be administered in terms of dosage forms, either separately or in association with one another and, in terms of administration times, either sequentially or simultaneously.

According to another embodiment, the compounds of the invention are not administered within the framework of a combined therapy with an additional therapeutic agent and/or active substance, in particular, the compounds of the invention are not administered within the framework of a combined therapy with an antibacterial agent.

Generally, for a pharmaceutical use, the compounds of the invention can be formulated in the form of a pharmaceutical preparation comprising at least one compound of the invention and at least one pharmaceutically acceptable excipient and optionally one or more other pharmaceutically active compounds.

By way of non-limiting examples, such a formulation can be in a form suitable for oral administration, parenteral administration (for example by intravenous, intramuscular or subcutaneous injection or by intravenous perfusion), for topical administration (including ocular), for administration by inhalation, by means of a skin patch, via an implant, via a suppository, etc. These suitable forms of administration, which may be solid, semi-solid or liquid depending on the method of administration, as well as the methods and supports, diluents and excipients to be used for their preparation, will be clear to a person skilled in the art; reference is made to the latest edition of Remington's Pharmaceutical Sciences.

Preferred, but not limiting, examples of such preparations include, tablets, pills, powders, lozenges, sachets, wafer capsules, elixirs, suspensions, emulsions, solutions, syrups, ointments, creams, lotions, soft and hard gelatin capsules, sterile injectable solutions and sterile packaged powders (which are generally reconstituted before use) for bolus administration and/or for continuous administration, which can be formulated with supports, excipients and diluents which are suitable per se for such formulations, such as lactose, dextrose, sucrose, sorbitol, mannitol, starches, gum acacia, calcium phosphate, alginates, tragacanth gum, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, food oils, vegetable and mineral oils or the suitable mixtures thereof. The formulations can optionally contain other substances commonly used in pharmaceutical formulations, such as lubricants, wetting agents, emulsifiers and suspension agents, dispersants, disintegrating agents, bulking agents, filling agents, preservatives, sweeteners, flavourings, flow regulators, mould release agents, etc. The compositions can also be formulated so as to ensure a quick, prolonged or delayed release of the one or more active compounds that they contain.

The pharmaceutical preparations of the invention are preferably in the form of unitary doses and can be suitably packaged, for example in a box, blister, bottle, sachet, ampoule or any other suitable single-dose or multiple-dose support or receptacle (which can be correctly labelled); optionally with one or more leaflets containing information on the product and/or instructions for use. Generally, these unitary doses contained between 1 and 1000 mg, and generally between 1 and 500 mg, preferably between 250 and 500 mg of at least one compound of the invention.

In practice, the effective dose to be administered depends on one or more parameters including, in particular, the equipment used for the administration, age, sex, height, weight, physical condition and degree of severity of the disorder to be treated.

In general, the active compound of the invention will be administered between 0.1 mg per kilogram and 5000 mg per kilogram of body weight, more often between 1 mg per kilogram and 2000 mg per kilogram of body weight, preferably between 1 and 100 mg per kilogram of body weight, for example approximately 1, 10, 100 mg per kilogram of body weight of the human patient per day, which can be administered in a single daily dose, divided into one or more daily doses, or essentially continuously, for example by using a drip perfusion.

### BRIEF DESCRIPTION OF THE FIGURES

FIG. 1 is a histogram showing the percentage of positive bacterial culture in the spleen assessed at 24 h, 48 h and 72 h.

FIG. 2 is a histogram showing the survival rate of mice after LPC (Caecal ligation and puncture) at 24 h, 48 h, 72 h and 96 h.

FIG. 3 is a histogram showing the decrease of temperature after LPC, monitored over 48 hours.

FIG. 4 is a histogram showing the weight (FIG. 4A) and weight loss (FIG. 4B) of mice (FIG. 4B) of mice after LPC at 24 h, 48 h, 72 h and 96 h.

FIG. 5 is a histogram showing the clinical score of mice after LPC at 24 h, 48 h, 72 h and 96 h.

FIG. 6 is a histogram showing the bacterial load in blood 24 hours after LPC, assessed by RT-PCR quantification of bacterial DNA (16S).

FIG. 7 is a histogram showing bacterial load in blood (FIG. 7A) and lungs (FIG. 7B), 5 days after infection with Pseudomonas aeruginosa.

### EXAMPLES

The present invention will be better understood on reading the following examples which illustrate the invention in a non-limiting manner.

### I. Synthesis of the Compounds of the Invention

1. Material and Methods

All the chemicals were obtained from commercial suppliers and users without further purification.

Thin layer chromatography was carried out on plastic sheets of TLC silica gel 60 F254 (layer thickness 0.2 mm) from Merck. Purification by column chromatography was carried out on the silica gel 60 (70-230 mesh ASTM, Merck). The melting points were determined either on a digital apparatus (Electrothermal IA 8103) and are not corrected, or on a WME Kofler bench (Wagner & Munz). The IR, ¹H, ¹⁹F and ¹³C NMR spectra confirmed the structure of all the compounds. The IR spectra were recorded on a Perkin Elmer Spectrum 100 FT-IR spectrometer and the NMR spectra were recorded, using CDCl₃, CD₃CN, D₂O or DMSO-d₆ as solvent, on a BRUKER AC 300 or 400 spectrometer at 300 or 400 MHz for the ¹H spectrum, 75 or 100 MHz for the ¹³C spectrum and 282 or 377 MHz for the ¹⁹F spectrum. The chemical shifts (δ) were expressed in parts per million with respect to the signal, indirectly (i) to CHCl₃ (δ 7.27) for ¹H and (ii) to CDCl₃ (δ 77.2) for ¹³C and directly (iii) to CFCl₃ (internal standard) (δ 0) for ¹⁹F. The chemical shifts are given in ppm and the multiplicities of peaks are designated as follows: s, singlet; br s, wide singlet; d, doublet; dd, doublet of doublet; t, triplet; q, quadruplet; quint, quintuplet; m, multiplet. High resolution mass spectra (HRMS) were obtained from the “Service central d'analyse de Solaize” (Centre national de la recherche scientifique) and have been recorded on a Waters spectrometer, using electrospray ionisation-TOF (ESI-TOF).

General Procedure

### Step 1: Synthesis of the Compound of Formula A-1

The compound of formula D (1.0 equiv.) is dissolved in dichloromethane. The nicotinamide of formula E (1.50 equiv.) and TMSOTf (1.55 equiv.) are added at ambient temperature. The mixture is heated with reflux and stirred until the reaction is achieved. The mixture is cooled to ambient temperature and filtered. The filtrate is concentrated to dryness to give tetraacetate A-1.

### Step 2: Synthesis of the Compound of Formula A-2

The tetraacetate A-1 is dissolved in methanol and cooled to −10° C. 4.6 M ammonia in methanol (3.0 equivalents) at −10° C. is added and the mixture is stirred at this temperature until the reaction is complete. Dowex HCR (H+) resin is added to a pH of 6-7. The reaction mixture is heated to 0° C. and filtered. The resin is washed with a mixture of methanol and acetonitrile. The filtrate is concentrated to dryness. The residue is dissolved in acetonitrile and concentrated to dryness. The residue is dissolved in acetonitrile to give a solution of the compound of formula A-2.

### Step 3: Synthesis of the Compound of Formula A-3

The crude solution of the compound of formula A-2 in acetonitrile is diluted with trimethyl phosphate (10.0 equivalents). The acetonitrile is distilled under vacuum and the mixture is cooled to −10° C. Phosphorus oxychloride (4,0 equivalents) is added at −10° C. and the mixture is stirred at −10° C. until the reaction is ended.

### Step 4 and 5: Synthesis of the Compound of Formula I-A

The mixture obtained in step 3 here above is hydrolysed by the addition of a 50/50 mixture of acetonitrile and water, followed by the addition of methyl tert-butyl ether. The mixture is filtered and the solid is dissolved in water. The aqueous solution is neutralised by the addition of sodium bicarbonate and extracted with dichloromethane. The aqueous layer is concentrated to dryness in order to give the crude compound of formula I-A, which is purified on a DOWEX 50wx8 column with elution in water followed by a silica gel chromatograph column.

### II. Biological Studies

### Example 1: In Vivo Efficacy of the Compound of Formula I-A in a Non-Lethal Model of Escherichia coli (E. coli) Induced Pneumonia

The purpose of this study is to evaluate the effect of pre-treatment, with an NAD precursor, on the spread of bacterial infection to the spleen in a mouse model of non-lethal Escherichia coli (E. coli) induced pneumonia.

1. Material and Methods

The administration of the compound at 185 mg/kg and the vehicle (physiological buffer) is performed intraperitoneally and/or intratracheally.

The compound of formula I-A (white powder) is dissolved in the vehicle. The solution is used at room temperature for a maximum of 1 day and freshly prepared for each new experiment.

The mice are weighed daily in order to adapt the volume of compound to be administered.

1.1. Escherichia coli Induced Pneumonia

Immediately before use, the E. coli culture was washed twice with 0.9% NaCl. After the second wash, the pellet was re-suspended in sterile saline and the dose calibrated by nephelometry.

Female BALB/c mice (20-24 g) were then inoculated using intratracheal insertion of a gavage needle (24 G) for injection of 75 μL of the bacterial suspension.

1.2. Administration of the Compounds

The compound of formula I-A and the vehicle are administered to the animals intraperitoneally and/or intratracheally. The injection of the compound of formula I-A is performed 24 hours prior to the infection with Escherichia coli. Simulated animals are given a physiological buffer by intraperitoneal administration.

1.3. Bacterial Load

At 24 hours, 48 hours, and 72 hours postoperatively, spleens from sacrificed animals were weighed and homogenized in 1 mL of saline. These solutions were then used for quantitative agar gel cultures during 24 hours of incubation at 37° C. Viable bacterial colony counts are expressed as Log 10 CFU per gram of organ.

2. Results and Discussion

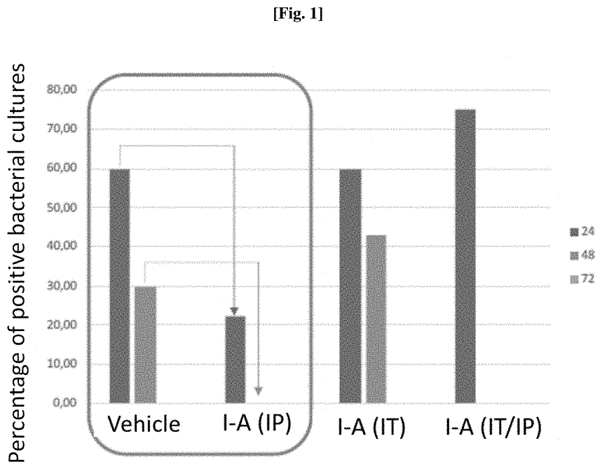

FIG. 1 shows the percentage of positive bacterial culture in the spleen assessed at 24 h, 48 h and 72 h. As shown, pre-treatment with the compound of formula I-A reduced the percentage of positive cultures in the spleen compared to untreated mice (control) after intraperitoneal administration. Notably, no bacteria were found in the spleen 48 h after intraperitoneal administration and 72 h after intratracheal administration.

3. Conclusion

After induction of non-lethal pneumonia by E. coli in mice, the spleens were collected and analyzed.

The percentage of animals with bacteria in the spleen after 24 hours was greatly reduced in the group treated with compound of formula I-A compared to the control group. Furthermore, no bacteria were found in the spleen of mice pre-treated with compound of formula I-A 48 hours after intraperitoneal administration while animals in the control group were positive.

Pre-treatment with compound of formula I-A prevented the spread of bacteria from the lungs to the spleen after intraperitoneal and/or intratracheal administration demonstrating a potential effect of compound of formula I-A in the prevention of sepsis during a bacterial infection.

### Example 2: In Vivo Evaluation of Compounds of Formula I-A and I-B on the Course of Sepsis in a Lethal Model of Caecal Ligation and Puncture

The purpose of this study is to evaluate the effect of administration of compounds according to the invention (I-A and I-B) on the survival rate of mice in a caecal ligation and puncture (LPC) induced sepsis model.

All procedures were performed in accordance with the Guide for the Care and Use of Laboratory Animals (revised 1996 and 2011, 2010/63/EU) and French laws.

1. Material and Methods

1.1. Protocol of the Study

The study consists of creating a sepsis model in mice by caecal ligation and puncture (LPC) and evaluating the impact of compounds I-A and I-B on the development of sepsis for 4 days.

The test compounds are administered at 185 mg/kg, intraperitoneally, immediately after the LPC procedure, and then once daily at 24, 48, and 72 hours. The remaining mice are euthanized at 96 hours post-LPC. Vehicle (physiological buffer) is used as a control and administered under the same conditions.

Three groups are formed and a total of 36 mice are included in the study:

Group 1: LPC+vehicle i.p. (n=12);

Group 2: LPC+compound I-A i.p. (n=12);

Group 3: LPC+compound 1-B i.p. (n=12).

In each group, survival, temperature, body weight and clinical score were assessed over the 4 days of the study. Bacterial load assessed at 24 hours after LPC.

1.1. Ligation and Puncture of the Caecum

Mice are anesthetized with 3% Vetoflurane. A laparotomy is performed to exteriorize the caecum. For induction of medium grade sepsis, the caecum is ligated between the distal pole and the base of the caecum. A through puncture with a 21-gauge needle is performed. A small amount of excreta is extruded from the mesenteric and antimesenteric penetration holes to ensure patency. The caecum is repositioned over the abdominal cavity. The peritoneum, fascia, abdominal musculature and then the skin are closed by applying simple sutures. The animals are resuscitated by injecting pre-warmed normal saline (37° C.; 5 ml per 100 g body weight) subcutaneously.

1.2. Administration of the Compounds

Administration of Compound I-A at 185 mg/kg, Compound I-B at 185 mg/kg and vehicle (physiological buffer) is performed intraperitoneally immediately after LPC surgery and then once daily at 24, 48 and 72 hours post LPC.

Preparation of the formulations: the powder of compounds I-A and I-B are dissolved in the vehicle. Storage conditions: The solution is used at room temperature for a maximum of 1 day and freshly prepared for each new experiment.

Mice are weighed daily to adapt the volume of compound to be administered.

1.3. Survival

Survival is assessed every 12 hours for the first two days, then once a day until euthanasia.

1.4. Temperature

Rectal temperature is checked every 2 to 4 hours for the first day, then twice for the second day.

1.5. Body Weight and Clinical Score

These two parameters are monitored once a day.

The clinical score is defined according to the criteria defined in Table 3:

TABLE 3
Murine sepsis score

| Score | 0 | 1 | 2 | 3 |
|---|---|---|---|---|
| Appearance | Smooth | Slightly rough | Most of the back is | Pilo erection |
| | coat | coat | ruffled | may or may not |
| | | | | be present, the |
| | | | | mouse appears |
| | | | | “puffy”. |
| Level of | Active | Active, avoids | The mouse activity | Activity is |
| consciousness | | standing | is noticeably | reduced. The |
| | | | slowed. The mouse | mouse only |
| | | | is still ambulatory | moves when |
| | | | | provoked, |
| | | | | movements have |
| | | | | a tremor |
| Activity | Normal | Suppression of | Suppressed activity. | No activity. The |
| | | eating, drinking | The mouse is | mouse is |
| | | or running | stationary and | stationary |
| | | | occasionally moves | |
| | | | to investigate | |
| Response to | Normal | Slow response | No response to | No response to |
| stimuli | | to auditory or | auditory stimulus; | auditory |
| | | tactile stimuli | moderate response | stimulus; slight |
| | | | to touch (moves a | response to |
| | | | few steps) | touch (no |
| | | | | locomotion) |
| Eyes | Open | Not fully open, | Eyes at least half | Eyes half closed |
| | | potential | closed, possibly | or more, |
| | | secretion | with secretions | possibly with |
| | | | | secretions |
| Breathing | Normal | Brief periods of | Labored, without | Brief periods of |
| quality | | labored | panting | labored |
| | | breathing | | breathing |
| | | | | Labored, |
| | | | | without panting |
| | | | | Labored with |
| | | | | intermittent |
| | | | | panting |

1.6. Bacterial Load

24 hours after surgery, mice were anesthetized with Vetoflurane and blood was collected from the retroorbital sinus to quantify the bacterial load. Bacterial DNA (16S) is extracted and quantified by RT-PCR.

2. Results and Discussion

1.1. Survival Rate

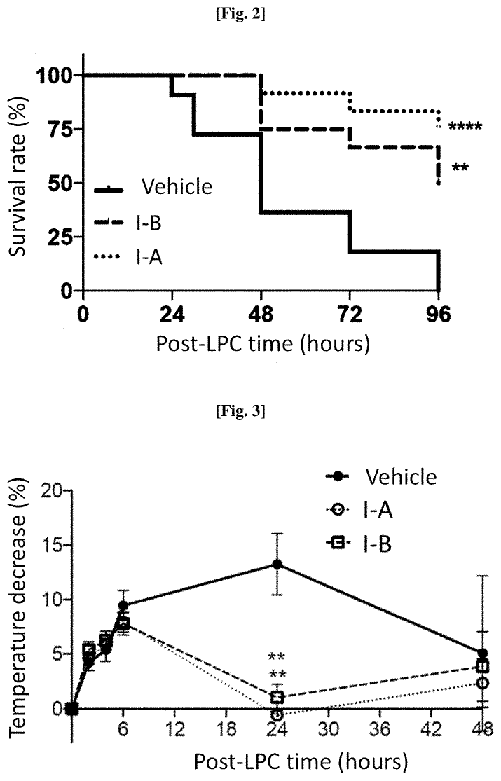

FIG. 2 shows the survival rate of mice after LPC at 24, 48, 72 and 96 hours. Table 4 specifies the number of surviving mice after LPC at 24, 48, 72 and 96 hours.

TABLE 4

| | Number of surviving mice | | |
|---|---|---|---|
| Time (h) | Vehicle | I-A | I-B |
| 24 | 11/11 | 12/12 | 12/12 |
| 48 | 5/11 | 11/12 | 9/12 |
| 72 | 3/11 | 10/12 | 8/12 |
| 96 | 0/11 | 9/12 | 6/12 |

The results in FIG. 2 and Table 4 show that the survival rate in mice treated with compound I-A or I-B is improved compared with the control group.

1.2. Temperature

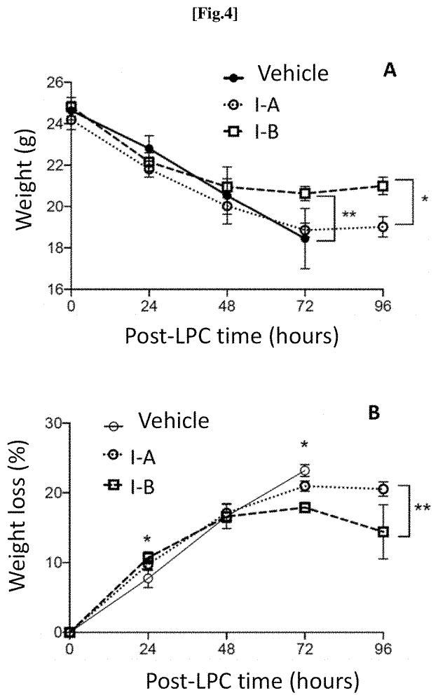

As shown in FIG. 3, a significant decrease in temperature is observed in the first 6 hours after LPC. Mice treated with compound I-A or 1-B regain their basal temperature 24 hours after surgery while the temperature of the control group continues to decrease.

1.3. Weight Loss

FIG. 4A shows the weight of mice after LPC at 24, 48, 72, and 96 hours, and FIG. 4B illustrates their weight loss over this period. Induced sepsis leads to a decrease in the weight of mice in all three groups. However, the weight loss is less in the group treated with compound I-B.

1.4. Clinical Score

FIG. 5 shows the clinical score of mice after LPC at 24, 48, 72, and 96 hours. The clinical score is very significantly improved in the groups treated with compound I-A or I-B, compared to the control group.

1.5. Bacterial Load

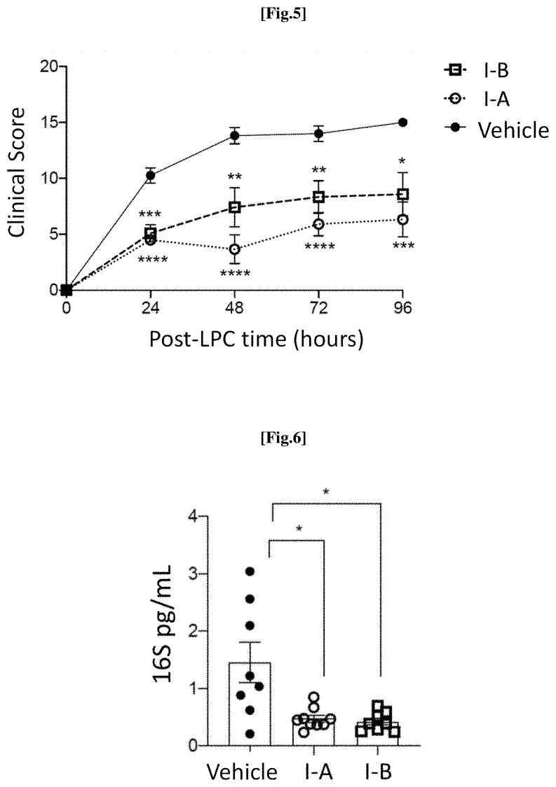

FIG. 6 shows the bacterial load in the blood, 24 hours after LPC. The bacterial load is significantly decreased in the groups treated with compound I-A or I-B, compared to the control group.

3. Conclusion

Treatment with compound I-A or I-B increased survival in treated mice in a lethal sepsis model. The treatment also minimized temperature drop, delayed infection progression and reduced bacterial load.

### Example 3: In Vivo Evaluation of Compound I-A in a Mouse Model of Respiratory Tract Infection Induced by Pseudomonas aeruginosa PAO₁

The purpose of this study is to evaluate the antibacterial effect of the administration of compound I-A in a lethal mouse model of Pseudomonas aeruginosa airway infection and its effect on sepsis.

1. Material and Methods

1.1. Protocol of the Study

The study consists in creating a model of respiratory infection in mice, by intranasal administration of 1.10⁴ CFU of Pseudomonas aeruginosa PAO₁, and to evaluate the effect of compound I-A on the bacterial load.

Compound I-A was administered at 185 mg/kg, intraperitoneally, 1 h after infection, and then once a day until euthanasia. The vehicle (physiological buffer) is used as a control and is administered under the same conditions. Ciprofloxacin is used as a reference compound and is administered under the same conditions.

Four groups (10 mice/group) are included in the study:
Vehicle group: PAO₁ infection+physiological buffer;
Ciprofloxacin group: PAO₁ infection+ciprofloxacin (2 mg/kg);
Group I-A: PAO₁ infection+compound I-A (185 mg/kg);
Group I-A+ciprofloxacin: PAO₁ infection+compound I-A (185 mg/kg)+ciprofloxacin (2 mg/kg).

In each group, the bacterial load in the blood and lungs is assessed on day 5 post infection.

1.1. Leukopenia and Infection

Leukopenia is induced by intraperitoneal injections of cyclophosphamide 4 days and 1 day before infection, with doses of 150 mg/kg and 100 mg/kg, respectively. P. aeruginosa PAO₁ was obtained from ATCC. One vial is thawed and diluted in sterile PBS on the day of infection. Mice are infected by intranasal administration of 1 10⁴ CFU of Pseudomonas aeruginosa PAO₁.

1.2. Administration of the Compounds

Compound I-A at 185 mg/kg and vehicle (physiological buffer) are administered intraperitoneally (i.p.) 1 hour after infection and then once a day until euthanasia.

Preparation of the formulations: The powder of compound I-A is dissolved in the vehicle. Storage conditions: The powder is stored at +4° C. until use. The solution should be used at room temperature for maximum 1 day and freshly prepared for each new experiment.

Mice are weighed regularly to adjust the volume of compound to be administered.

For reference, ciprofloxacin is also administered at sub-active dose (2 mg/kg) to mice intraperitoneally under the same conditions.

A co-administration of ciprofloxacin and compound I-A is also performed, under the same conditions.

1.3. Bacterial Load

Bacterial load is assessed in blood and lungs on day 5. Quantitative cultures on agar gel are performed during 24 hours of incubation at 37° C. Viable bacterial colony counts are expressed as Log 10 CFU per mL of blood or per gram of organ.

2. Results and Discussion

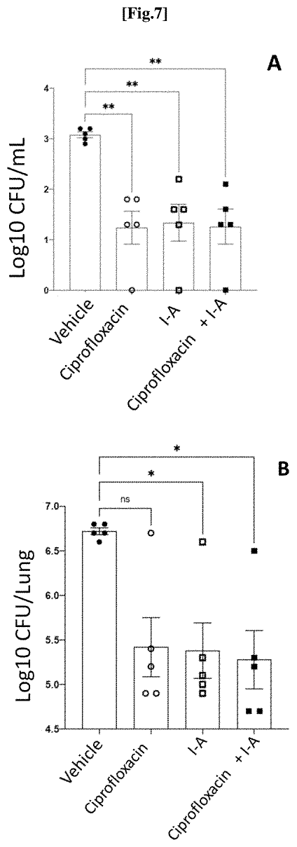

FIG. 7 shows the bacterial load in blood (FIG. 7A) and lungs (FIG. 7B), 5 days after infection with Pseudomonas aeruginosa.

Evaluation of the pulmonary bacterial load showed an antibacterial effect of treatment with compound I-A, ciprofloxacin, or a combination of ciprofloxacin and compound I-A. All 3 treatment groups showed significant efficacy compared with the vehicle group. Blood bacterial load results showed that P. aeruginosa triggered sepsis in all 4 groups. The three treated groups showed a reduced bacterial load compared to the vehicle group by approximately 2 log.

Thus, treatment with compound I-A limited the bacterial load during infection and limited sepsis.

1-11. (canceled)

12. A method for treating a bacterial infection in a subject in need thereof, said method comprising administering to said subject a therapeutically effective quantity of at least one compound of formula (I) 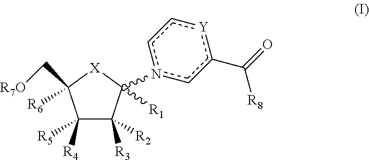
or a pharmaceutically acceptable salt and/or solvate thereof, wherein:
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from P(O)R₉R₁₀, P(S)R₉R₁₀ and 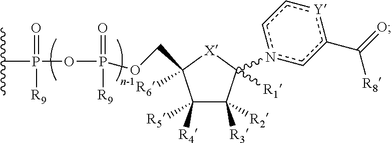
wherein
R₉, and R₁₀ are selected, independently of one another, from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, (C₅-C₁₂)-aryl-(C₁-C₈)-alkyl, (C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}C(O)R₁₂; wherein:
R₁₁ is selected from C₁-C₁₀ alkyl, C₃-C₁₀ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁-C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; P(O)(OH)OP(O)(OH)₂; and an internal or external counter-ion;
R₁₂ is selected from C₁-C₁₀ alkyl, hydroxy, C₁-C₁₀ alkoxy, C₂-C₈ alkenyloxy, C₂-C₈ alkynyloxy, halo(C₂-C₁₀)-alkoxy, C₃-C₁₀ cycloalkoxy, C₃-C₁₀ heterocycloalkyloxy, C₅-C₁₂ aryloxy, (C₁-C₄)-alkyl-(C₅-C₁₂)-aryloxy, (C₅-C₁₂)-aryl-(C₁-C₄)-alkyloxy and C₅-C₁₂ heteroaryloxy; wherein said aryl or heteroaryl groups are optionally substituted by one or two groups selected from halogen, trifluoromethyl, C₁-C₆ alkyl, C₁-C₆ alkoxy and cyano;
R₁₃ and R₁₄ are selected independently from H, C₁-C₈ alkyl and (C₁-C₈)-alkyl-(C₅-C₁₂)-aryl;
R_{α} and R_{α′} are selected independently from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen, nitro and cyano;
or R₉ and R₁₀ with the phosphorus atoms to which they are bonded, form a 6-member-ring, wherein —R₉-R₁₀— represents —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆ alkoxy and cyano;
X′ is selected from O, CH₂, S, Se, CHF, CF₂ and C═CH₂;
R_{1′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{8′} is selected from H, OR, NHR_{15′}, NR_{15′}R_{16′}, NH—NHR_{15′}, SH, CN, N₃ and halogen; wherein R_{15′} and R_{16′} are selected, independently of one another, from H, C₁-C₈ alkyl and C₁-C₈ alkyl-aryl;
Y′ selected from CH, CH₂, C(CH₃)₂ and CCH₃;
n is an integer selected from 1 to 3; [symbol]
represents a single or a double bond according to Y′; and [symbol]
represents the alpha or beta anomer according to the position of R_{1′};
R₈ is selected from H, OR, NHR₁₅, NR₁₅R₁₆, NH—NHR₁₅, SH, CN, N₃ and halogen; wherein R is selected from H and C₁-C₈ alkyl, and R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
Y is selected from CH, CH₂, C(CH₃)₂ and CCH₃; [symbol]
represents a single or a double bond according to Y; and [symbol]
represents the alpha or beta anomer according to the position of R₁.

13. The method according to claim 12, wherein X represents oxygen.

14. The method according to claim 12, wherein R₁ and R₆ each represent hydrogen.

15. The method according to claim 12, wherein R₂, R₃, R₄ and R₅ each represent, independently of one another, hydrogen or OH.

16. The method according to claim 12, wherein Y represents CH or CH₂.

17. The method according to claim 12, wherein R₇ represents P(O)(OH)₂.

18. The method according to claim 12, wherein R₇ represents 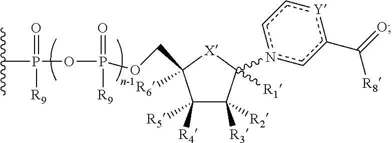
wherein;
R₉ is OH or OR₁₁, wherein R₁₁ is as defined in formula (I) and
X′ is oxygen;
R_{1′} and R_{6′} each represent hydrogen;
R_{2′}, R_{3′}, R_{4′} and R_{5′} are independently selected from hydrogen and OH;
R_{8′} is NH₂;
Y′ is selected from CH and CH₂;
n is equal to 2; [symbol]
represents a single or a double bond according to Y′; and [symbol]
represents the alpha or beta anomer according to the position de R_{1′}.

19. The method according to claim 12, wherein the compound of formula (I) is selected from: 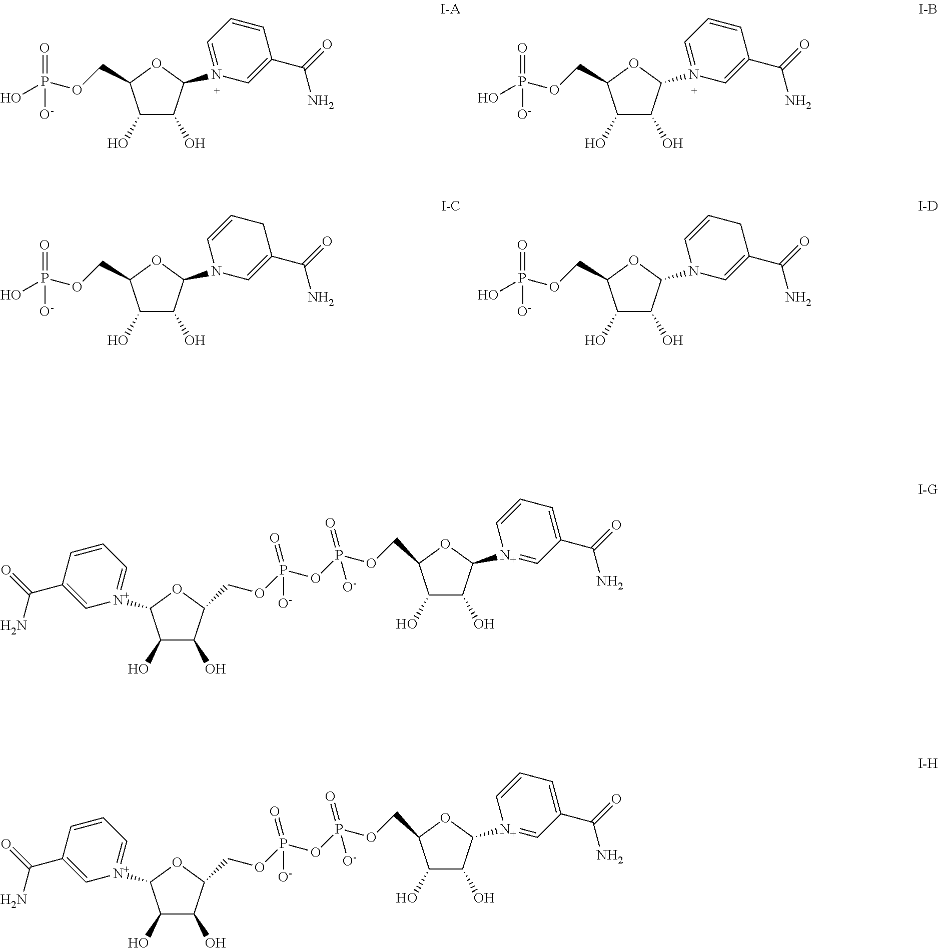 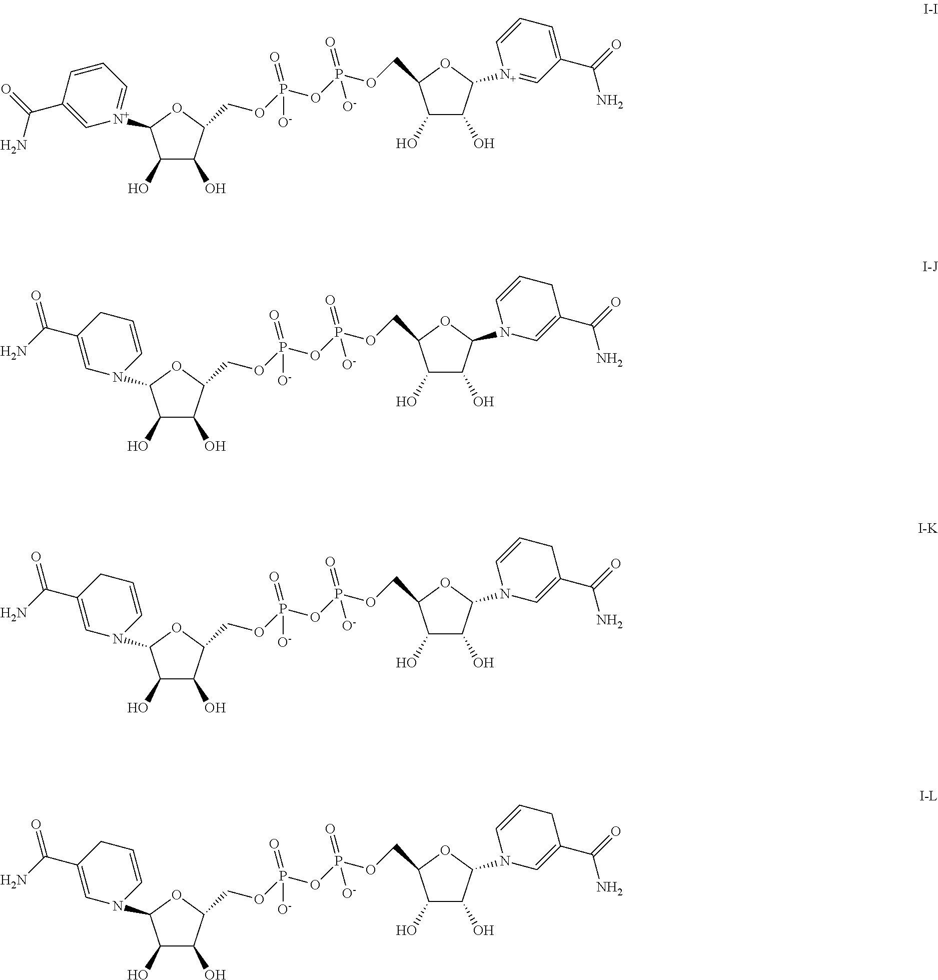
or a pharmaceutically acceptable salt and/or solvate thereof.

20. The method according to claim 12, wherein the bacterial infection is caused by at least one bacterium of the genus selected from aerobic Gram-positive bacteria; Gram-negative enterobacteria; Gram-negative bacilli; Gram-negative anaerobic bacteria; Gram-positive anaerobic bacteria; mycobacteria and pathogens involved in sexually transmitted infections.

21. The method according to claim 20, wherein the aerobic Gram-positive bacteria are selected from the group consisting of Streptococcus, Staphylococcus, Enterococcus and Bacillus; the Gram-negative enterobacteria are selected from the group consisting of Escherichia coli, Klebsiella pneumonia, Enterobacter aerogenes, Enterobacter cloacae, Proteus vulgaris, Shigella flexneri, Serratia marcescens, Citrobacter freundii, Yersinia enterocolitica and Salmonella enteritidis; the Gram-negative bacilli are selected from the group consisting of Pseudomonas aeruginosa, Acinetobacter baumannii, Burkholderia cepacia and Stenotrophomonas maltophilia; the Gram-negative anaerobic bacteria are selected from the group consisting of Bacteroides, Fusobacterium and Eubacterium; the Gram-positive anaerobic bacteria are selected from the group consisting of Propionibacterium, Peptococcus, Clostridium, Peptostreptococcus and Veillonella; the mycobacteria are selected from the group consisting of Mycobacterium leprae and Mycobacterium tuberculosis; and wherein the pathogens involved in sexually transmitted infections are selected from the group consisting of Neisseria, Haemophilus, Chlamydia and Mycoplasma.

21. The method according to claim 12, wherein the bacterial infection is selected from bacterial skin and soft tissue infections, sexually transmitted bacterial infections, tetanus, typhoid, tuberculosis, cholera, diphtheria, syphilis, salmonella, pulmonary bacterial infections or sepsis.

22. The method according to claim 12, wherein the bacterial infection is sepsis.
